(19) United States
(12) Patent Application Publication
(10) Pub. No.: US 2024/0182832 A1
(43) Pub. Date: Jun. 6, 2024

(54) SINGLE VESSEL MULTI-ZONE BIOREACTOR FOR SIMULTANEOUS CULTURE OF MULTIPLE MICROBIAL STRAINS

(71) Applicants: ROYAL MELBOURNE INSTITUTE OF TECHNOLOGY, Carlton, Victoria (AU); CITY UNIVERSITY OF HONG KONG, Hong Kong (CN)

(72) Inventors: Srinivas METTU, Melbourne, Victoria (AU); Namita ROY CHOUDHURY, Melbourne, Victoria (AU); Naba Kumar DUTTA, Melbourne, Victoria (AU); Carol Sze Ki LIN, Hong Kong (CN); Zubeen HATHI, Maharashtra (IN)

(21) Appl. No.: 18/284,884

(22) PCT Filed: Mar. 30, 2022
(86) PCT No.: PCT/AU2022/050290

§ 371 Date: Sep. 29, 2023

(30) Foreign Application Priority Data
Mar. 30, 2021 (AU) 2021900939

(51) Int. Cl. C12M 1/00; C12M 1/12; C12M 1/34; C12N 1/20

(52) U.S. Cl. CPC C12M 23/34; C12M 25/14; C12M 41/26; C12M 41/34; C12M 47/02; C12N 1/20

(57) ABSTRACT

A single vessel multi-zone bioreactor for simultaneously culturing multiple microbial strains comprising multiple culturing zones arranged sequentially, each culturing zone comprising a porous hydrogel, and the use thereof for producing optionally encapsulated probiotics.

### TECHNICAL FIELD

The invention disclosed herein resides in the field of chemical engineering for microbiological applications. More specifically, the present invention relates to bioreactors for fermentation, or for culturing microbial strains, and to methods for producing food additives, supplements or medications comprising microbial strains.

### BACKGROUND

The following discussion of the background to the invention is intended to facilitate an understanding of the present invention only. The discussion is not an acknowledgement or admission that any of the material referred to is or was part of the common general knowledge as at the priority date of the application.

Recent literature suggests that severely malnourished children do not recover to full health even after they are fed well later in their lives.¹⁻⁶ Their brains did not develop to their full potential earlier in life, and the children were more susceptible to diseases in the later stages of their lives.³⁻⁵ It has been clearly identified that the optimal functioning of the gut requires a specific microbial community or microbiome, because its coupling with suitable changes in the host's dietary habits generates the required energy for the host to deliver health benefits.

Restoration of the gut microbiome has the potential to offer malnourished children healthier lives. However, existing probiotic bacteria products available in the market contain only a few easy-to-grow strains from the Lactobacillus and Bifidobacterium genera which constitute only a small fraction of the gut microbiome.

To restore the overall loss in microbial diversity, caused either due to gut dysbiosis or malnutrition, it is essential to reconstitute the gut microflora by administering multiple strains of aerobic and anaerobic bacteria that belong to other phyla of human gut microbes.

The conventional practice of growing multiple strains of bacteria is to culture them in separate bioreactors using liquid media for growth as they require different nutrients, growth media and oxygen levels. However, growing each strain in a different bioreactor makes the production process complicated and costly to maintain, resulting in a probiotic product bearing a high cost that is not affordable for malnourished children in underdeveloped and developing countries.

To tackle this issue and to reduce the cost of production, there is a need to provide bioreactors that enable the growth of multiple strains of aerobic and anaerobic bacterial species simultaneously.

It is against this background that the present invention has been developed.

### SUMMARY OF THE INVENTION

In one embodiment, the disclosure of the present invention provides a single vessel multi-zone bioreactor for simultaneously culturing multiple microbial strains comprising; multiple culturing zones, wherein;
the multiple culturing zones are arranged sequentially so as to provide a gradient in pH, from lower pH to higher pH, or from acidic pH to basic pH, or from higher pH to lower pH, or from basic pH to acidic pH; and/or
the multiple culturing zones are arranged sequentially so as to provide a gradient in oxygen levels, from a higher partial pressure of oxygen to a lower partial pressure of oxygen, or from aerobic conditions to anaerobic conditions, or from a lower partial pressure of oxygen to a higher partial pressure of oxygen, or from anaerobic conditions to aerobic conditions.

In some embodiments of the single vessel multi-zone bioreactor, the multiple culturing zones arranged sequentially each comprise, a porous hydrogel, and a liquid culturing media, wherein, upon inoculation of each culturing zone with a microbial strain, and through to completion of the culturing process, each liquid culturing media may be referred to as a broth.

In some embodiments of the single vessel multi-zone bioreactor, the multiple culturing zones are arranged sequentially to provide a sequence of at least three culturing zones; wherein the multiple culturing zones each comprise, a porous hydrogel, and a liquid culturing media.

In some embodiments of the single vessel multi-zone bioreactor, the multiple culturing zones are arranged sequentially to provide a sequence of culturing zones wherein the number of sequentially arranged culturing zones in the sequence is selected from the group consisting of; 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60 sequentially arranged culturing zones.

In some embodiments, the single vessel multi-zone bioreactor is adapted to be operated in batch culture mode, or fed-batch culture mode, or continuous culture mode, to produce at least 100 ml per day per culturing zone of optimal microbial culture broth.

In some embodiments, the single vessel multi-zone bioreactor is adapted to be operated in batch culture mode, or fed-batch culture mode, or continuous culture mode, to produce a volume per day, per culturing zone of optimal microbial culture broth selected from the group consisting of; 0.1 L, 0.2 L, 0.3 L, 0.4 L, 0.5 L, 0.6 L, 0.7 L, 0.8 L, 0.9 L, 1 L, 1.1 L, 1.2 L, 1.3 L, 1.4 L, 1.5 L, 1.6 L, 1.7 L, 1.8 L, 1.9 L, 2 L, 2.1 L, 2.2 L, 2.3 L, 2.4 L, 2.5 L, 2.6 L, 2.7 L, 2.8 L, 2.9 L, 3 L, 3.1 L, 3.2 L, 3.3 L, 3.4 L, 3.5 L, 3.6 L, 3.7 L, 3.8 L, 3.9 L, 4 L, 4.1 L, 4.2 L, 4.3 L, 4.4 L, 4.5 L, 4.6 L, 4.7 L, 4.8 L, 4.9 L, 5 L, 5.1 L, 5.2 L, 5.3 L, 5.4 L, 5.5 L, 5.6 L, 5.7 L, 5.8 L, 5.9 L, 6 L, 6.1 L, 6.2 L, 6.3 L, 6.4 L, 6.5 L, 6.6 L, 6.7 L, 6.8 L, 6.9 L, 7 L, 7.1 L, 7.2 L, 7.3 L, 7.4 L, 7.5 L, 7.6 L, 7.7 L, 7.8 L, 7.9 L, 8 L, 8.1 L, 8.2 L, 8.3 L, 8.4 L, 8.5 L, 8.6 L, 8.7 L, 8.8 L, 8.9 L, 9 L, 9.1 L, 9.2 L, 9.3 L, 9.4 L, 9.5 L, 9.6 L, 9.7 L, 9.8 L, 9.9 L, and 10 L.

In some embodiments, an optimal microbial culture broth is one in which the Dry Cell Weight (DCW) is at least 0.3 g/L; more preferably, in which the Dry Cell Weight (DCW) is within the range of 0.3 to 4.0 g/L; most preferably in which the Dry Cell Weight (DCW) is selected from the group consisting of; 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.1 g/L, 2.2 g/L, 2.3 g/L, 2.4 g/L, 2.5 g/L, 2.6 g/L, 2.7 g/L, 2.8 g/L, 2.9 g/L, 3.0 g/L, 3.1 g/L, 3.2 g/L, 3.3 g/L, 3.4 g/L, 3.5 g/L, 3.6 g/L, 3.7 g/L, 3.8 g/L, 3.9 g/L, and 4.0 g/L.

In some embodiments, the single vessel multi-zone bioreactor is adapted to be operated in fed-batch culture mode, or continuous culture mode, wherein each respective microbial culture broth is removed from each corresponding culturing zone at the point in time that each respective microbial culture broth within each corresponding culturing zone reaches a stationary phase.

In some embodiments, a stationary phase in microbial growth is observed within a culturing zone of the single vessel multi-zone bioreactor, within a period of time falling within the range of about 6 hours to about 72 hours, or within a period of time selected from the group consisting of; 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, and 72 hours.

In some embodiments, the porosity of the hydrogel in each culturing zone differs from the porosity of the hydrogel in each adjacent culturing zone; and/or
the surface chemistry, and/or composition of the hydrogel in each culturing zone differs from the surface chemistry, and/or composition of the hydrogel in each adjacent culturing zone; and/or
the water retention of the hydrogel in each culturing zone differs from the water retention of the hydrogel in each adjacent culturing zone; and/or
the Young's modulus and/or the toughness of the hydrogel in each culturing zone differs from the Young's modulus and/or the toughness of the hydrogel in each adjacent culturing zone.

In some embodiments, the porosity of the hydrogel in each culturing zone differs from the porosity of the hydrogel in each adjacent culturing zone, such that the sequence of sequentially arranged culturing zones provides a hydrogel porosity gradient; and/or
the surface chemistry of the hydrogel in each culturing zone differs from the surface chemistry of the hydrogel in each adjacent culturing zone, such that the sequence of sequentially arranged culturing zones provides a hydrogel surface hydrophilicity gradient; and/or
the water retention of the hydrogel in each culturing zone differs from the water retention of the hydrogel in each adjacent culturing zone, such that the sequence of sequentially arranged culturing zones provides a hydrogel water retention gradient; and/or
the Young's modulus and/or the toughness of the hydrogel in each culturing zone differs from the Young's modulus and/or the toughness of the hydrogel in each adjacent culturing zone, such that the sequence of sequentially arranged culturing zones provides a Young's modulus and/or toughness gradient.

In some embodiments, the hydrogel porosity gradient comprises;
an increasing porosity gradient, where the porosity of the hydrogel in each culturing zone is greater than the porosity of the hydrogel in the previous culturing zone in the sequence of sequentially arranged culturing zones; or
a decreasing porosity gradient, where the porosity of the hydrogel in each culturing zone is less than the porosity of the hydrogel in the previous culturing zone in the sequence of sequentially arranged culturing zones.

In some embodiments, the hydrogel surface hydrophilicity gradient comprises;
an increasing hydrogel surface hydrophilicity gradient, where the hydrophilicity of the hydrogel in each culturing zone is greater than the hydrophilicity of the hydrogel in the previous culturing zone in the sequence of sequentially arranged culturing zones; or
a decreasing hydrogel surface hydrophilicity gradient, where the hydrophilicity of the hydrogel in each culturing zone is less than the hydrophilicity of the hydrogel in the previous culturing zone in the sequence of sequentially arranged culturing zones.

In some embodiments, the hydrogel water retention gradient comprises;
an increasing hydrogel water retention gradient, where the water retention of the hydrogel in each culturing zone is greater than the water retention of the previous culturing zone in the sequence of sequentially arranged culturing zones; or
a decreasing hydrogel water retention gradient, where the water retention of the hydrogel in each culturing zone is less than the water retention of the previous culturing zone in the sequence of sequentially arranged culturing zones.

In some embodiments of the single vessel multi-zone bioreactor, the multiple culturing zones arranged sequentially comprise porous hydrogels, wherein the porous hydrogels comprise one or more substances selected from the group consisting of; polysaccharides, cellulose, cellulose nano fibers, cellulose derivatives, methyl cellulose, alginates, dextran, hyaluronan, hyaluronates, agar, agarose, agaropectin, chitin, chitosan, gelatin, collagen, poly(lactic-co-glycolic acid), poly(e-caprolactone), poly(glycolic acid), PLA, PVA, PAM, PEG, PEGDA, PHEMA, proteins, polypeptides, biomimetic proteins, whey proteins, soy proteins, poly(lysine), elastins, elastin mimetic proteins, resilin, resilin mimetic proteins, insulin, trypsin, catalase, deoxyribonuclease, lysozymes, amyloids, β-galactosidase, silk fibroin, fibrinogen, including pharmaceutically acceptable derivatives and/or salts of any of the aforementioned substances.

In some embodiments of the single vessel multi-zone bioreactor, the multiple culturing zones arranged sequentially are nested, in sequence, such that the next culturing zone in the sequence, is nested within the previous culturing zone in the sequence; or
the multiple culturing zones arranged sequentially are nested, in sequence, such that the previous culturing zone in the sequence, is nested within the next culturing zone in the sequence.

In some embodiments of the single vessel multi-zone bioreactor, each culturing zone is separated from adjacent culturing zones by a porous membrane, capable of preventing transmigration of cultured microbial strains into adjacent culturing zones, while allowing biochemical communication between adjacent culturing zones, including allowing the transfer of culturing media and microbial metabolites between adjacent culturing zones; preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm.

In some embodiments, the porous membrane employed in various embodiments of the invention has a pore size selected from the group consisting of 0.01 μm, 0.02 μm, 0.03 μm, 0.04 μm, 0.05 μm, 0.06 μm, 0.07 μm, 0.08 μm, 0.09 μm, 0.1 μm, 0.11 μm, 0.12 μm, 0.13 μm, 0.14 μm, 0.15 μm, 0.16 μm, 0.17 μm, 0.18 μm, 0.19 μm, 0.2 μm, 0.21 μm, 0.22 μm, 0.23 μm, 0.24 μm, 0.25 μm, 0.26 μm, 0.27 μm, 0.28 μm, 0.29 μm, 0.3 μm, 0.31 μm, 0.32 μm, 0.33 μm, 0.34 μm, 0.35 μm, 0.36 μm, 0.37 μm, 0.38 μm, 0.39 μm, 0.4 μm, 0.41 μm, 0.42 μm, 0.43 μm, 0.44 μm, 0.45 μm, 0.46 μm, 0.47 μm, 0.48 μm, 0.49 μm, 0.5 μm, 0.51 μm, 0.52 μm, 0.53 μm, 0.54 μm, 0.55 μm, 0.56 μm, 0.57 μm, 0.58 μm, 0.59 μm, 0.6 μm, 0.61 μm, 0.62 μm, 0.63 μm, 0.64 μm, 0.65 μm, 0.66 μm, 0.67 μm, 0.68 μm, 0.69 μm, 0.7 μm, 0.71 μm, 0.72 μm, 0.73 μm, 0.74 μm, 0.75 μm, 0.76 μm, 0.77 μm, 0.78 μm, 0.79 μm, 0.8 μm, 0.81 μm, 0.82 μm, 0.83 μm, 0.84 μm, 0.85 μm, 0.86 μm, 0.87 μm, 0.88 μm, 0.89 μm, 0.9 μm, 0.91 μm, 0.92 μm, 0.93 μm, 0.94 μm, 0.95 μm, 0.96 μm, 0.97 μm, 0.98 μm, 0.99 μm, 1 μm, 1.01 μm, 1.02 μm, 1.03 μm, 1.04 μm, 1.05 μm, 1.06 μm, 1.07 μm, 1.08 μm, 1.09 μm, 1.1 μm, 1.11 μm, 1.12 μm, 1.13 μm, 1.14 μm, 1.15 μm, 1.16 μm, 1.17 μm, 1.18 μm, 1.19 μm, 1.2 μm, 1.21 μm, 1.22 μm, 1.23 μm, 1.24 μm, 1.25 μm, 1.26 μm, 1.27 μm, 1.28 μm, 1.29 μm, 1.3 μm, 1.31 μm, 1.32 μm, 1.33 μm, 1.34 μm, 1.35 μm, 1.36 μm, 1.37 μm, 1.38 μm, 1.39 μm, 1.4 μm, 1.41 μm, 1.42 μm, 1.43 μm, 1.44 μm, 1.45 μm, 1.46 μm, 1.47 μm, 1.48 μm, 1.49 μm, 1.5 μm, 1.51 μm, 1.52 μm, 1.53 μm, 1.54 μm, 1.55 μm, 1.56 μm, 1.57 μm, 1.58 μm, 1.59 μm, 1.6 μm, 1.61 μm, 1.62 μm, 1.63 μm, 1.64 μm, 1.65 μm, 1.66 μm, 1.67 μm, 1.68 μm, 1.69 μm, 1.7 μm, 1.71 μm, 1.72 μm, 1.73 μm, 1.74 μm, 1.75 μm, 1.76 μm, 1.77 μm, 1.78 μm, 1.79 μm, 1.8 μm, 1.81 μm, 1.82 μm, 1.83 μm, 1.84 μm, 1.85 μm, 1.86 μm, 1.87 μm, 1.88 μm, 1.89 μm, 1.9 μm, 1.91 μm, 1.92 μm, 1.93 μm, 1.94 μm, 1.95 μm, 1.96 μm, 1.97 μm, 1.98 μm, 1.99 μm, 2 μm, 2.01 μm, 2.02 μm, 2.03 μm, 2.04 μm, 2.05 μm, 2.06 μm, 2.07 μm, 2.08 μm, 2.09 μm, 2.1 μm, 2.11 μm, 2.12 μm, 2.13 μm, 2.14 μm, 2.15 μm, 2.16 μm, 2.17 μm, 2.18 μm, 2.19 μm, 2.2 μm, 2.21 μm, 2.22 μm, 2.23 μm, 2.24 μm, 2.25 μm, 2.26 μm, 2.27 μm, 2.28 μm, 2.29 μm, 2.3 μm, 2.31 μm, 2.32 μm, 2.33 μm, 2.34 μm, 2.35 μm, 2.36 μm, 2.37 μm, 2.38 μm, 2.39 μm, 2.4 μm, 2.41 μm, 2.42 μm, 2.43 μm, 2.44 μm, 2.45 μm, 2.46 μm, 2.47 μm, 2.48 μm, 2.49 μm, 2.5 μm, 2.51 μm, 2.52 μm, 2.53 μm, 2.54 μm, 2.55 μm, 2.56 μm, 2.57 μm, 2.58 μm, 2.59 μm, 2.6 μm, 2.61 μm, 2.62 μm, 2.63 μm, 2.64 μm, 2.65 μm, 2.66 μm, 2.67 μm, 2.68 μm, 2.69 μm, 2.7 μm, 2.71 μm, 2.72 μm, 2.73 μm, 2.74 μm, 2.75 μm, 2.76 μm, 2.77 μm, 2.78 μm, 2.79 μm, 2.8 μm, 2.81 μm, 2.82 μm, 2.83 μm, 2.84 μm, 2.85 μm, 2.86 μm, 2.87 μm, 2.88 μm, 2.89 μm, 2.9 μm, 2.91 μm, 2.92 μm, 2.93 μm, 2.94 μm, 2.95 μm, 2.96 μm, 2.97 μm, 2.98 μm, 2.99 μm, 3 μm, 3.01 μm, 3.02 μm, 3.03 μm, 3.04 μm, 3.05 μm, 3.06 μm, 3.07 μm, 3.08 μm, 3.09 μm, 3.1 μm, 3.11 μm, 3.12 μm, 3.13 μm, 3.14 μm, 3.15 μm, 3.16 μm, 3.17 μm, 3.18 μm, 3.19 μm, 3.2 μm, 3.21 μm, 3.22 μm, 3.23 μm, 3.24 μm, 3.25 μm, 3.26 μm, 3.27 μm, 3.28 μm, 3.29 μm, 3.3 μm, 3.31 μm, 3.32 μm, 3.33 μm, 3.34 μm, 3.35 μm, 3.36 μm, 3.37 μm, 3.38 μm, 3.39 μm, 3.4 μm, 3.41 μm, 3.42 μm, 3.43 μm, 3.44 μm, 3.45 μm, 3.46 μm, 3.47 μm, 3.48 μm, 3.49 μm, 3.5 μm, 3.51 μm, 3.52 μm, 3.53 μm, 3.54 μm, 3.55 μm, 3.56 μm, 3.57 μm, 3.58 μm, 3.59 μm, 3.6 μm, 3.61 μm, 3.62 μm, 3.63 μm, 3.64 μm, 3.65 μm, 3.66 μm, 3.67 μm, 3.68 μm, 3.69 μm, 3.7 μm, 3.71 μm, 3.72 μm, 3.73 μm, 3.74 μm, 3.75 μm, 3.76 μm, 3.77 μm, 3.78 μm, 3.79 μm, 3.8 μm, 3.81 μm, 3.82 μm, 3.83 μm, 3.84 μm, 3.85 μm, 3.86 μm, 3.87 μm, 3.88 μm, 3.89 μm, 3.9 μm, 3.91 μm, 3.92 μm, 3.93 μm, 3.94 μm, 3.95 μm, 3.96 μm, 3.97 μm, 3.98 μm, 3.99 μm, 4 μm, 4.01 μm, 4.02 μm, 4.03 μm, 4.04 μm, 4.05 μm, 4.06 μm, 4.07 μm, 4.08 μm, 4.09 μm, 4.1 μm, 4.11 μm, 4.12 μm, 4.13 μm, 4.14 μm, 4.15 μm, 4.16 μm, 4.17 μm, 4.18 μm, 4.19 μm, 4.2 μm, 4.21 μm, 4.22 μm, 4.23 μm, 4.24 μm, 4.25 μm, 4.26 μm, 4.27 μm, 4.28 μm, 4.29 μm, 4.3 μm, 4.31 μm, 4.32 μm, 4.33 μm, 4.34 μm, 4.35 μm, 4.36 μm, 4.37 μm, 4.38 μm, 4.39 μm, 4.4 μm, 4.41 μm, 4.42 μm, 4.43 μm, 4.44 μm, 4.45 μm, 4.46 μm, 4.47 μm, 4.48 μm, 4.49 μm, 4.5 μm, 4.51 μm, 4.52 μm, 4.53 μm, 4.54 μm, 4.55 μm, 4.56 μm, 4.57 μm, 4.58 μm, 4.59 μm, 4.6 μm, 4.61 μm, 4.62 μm, 4.63 μm, 4.64 μm, 4.65 μm, 4.66 μm, 4.67 μm, 4.68 μm, 4.69 μm, 4.7 μm, 4.71 μm, 4.72 μm, 4.73 μm, 4.74 μm, 4.75 μm, 4.76 μm, 4.77 μm, 4.78 μm, 4.79 μm, 4.8 μm, 4.81 μm, 4.82 μm, 4.83 μm, 4.84 μm, 4.85 μm, 4.86 μm, 4.87 μm, 4.88 μm, 4.89 μm, 4.9 μm, 4.91 μm, 4.92 μm, 4.93 μm, 4.94 μm, 4.95 μm, 4.96 μm, 4.97 μm, 4.98 μm, 4.99 μm, and 5 μm.

In some embodiments of the single vessel multi-zone bioreactor, each culturing zone is separated from adjacent culturing zones by a casting mould, wherein each casting mould comprises at least one aperture, allowing biochemical communication between adjacent culturing zones, including allowing the transfer of culturing media and microbial metabolites between adjacent culturing zones; optionally wherein;
the apertures comprise a porous membrane, capable of preventing transmigration of cultured microbial strains into adjacent culturing zones; most preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm; and/or
the casting moulds are fabricated from a sterilizable material such as but not limited to glass, porcelain, polypropylene (PP), Teflon or any fluoropolymer, stainless steel, other metals, or any pharmaceutically acceptable or food grade polymer; and/or the casting moulds are manufactured via 3D printing.

In some embodiments of the single vessel multi-zone bioreactor, the single vessel is a cylindrical vessel, and the multiple culturing zones of the bioreactor comprise a sequence of horizontally adjacent, nested concentric cylindrical culturing zones.

In some embodiments of the single vessel multi-zone bioreactor, the sequence of horizontally adjacent, nested concentric cylindrical culturing zones comprises;
an outermost culturing zone of lower pH, an innermost culturing zone of higher pH, and one or more intermediate culturing zones of intermediate pH, so as to provide a gradient in pH, from lower pH to higher pH, moving in sequence from the outermost culturing zone to the innermost culturing zone; and/or
an outermost culturing zone of acidic pH, an innermost culturing zone of basic pH, and one or more intermediate culturing zones of intermediate pH, so as to provide a gradient in pH, from acidic pH to basic pH, moving in sequence from the outermost culturing zone to the innermost culturing zone; and/or
an outermost culturing zone comprising a higher partial pressure of oxygen, an innermost culturing zone comprising a lower partial pressure of oxygen, and one or more intermediate culturing zones comprising an intermediate partial pressure of oxygen, so as to provide a gradient in partial pressures of oxygen, from higher partial pressures of oxygen to lower partial pressures of oxygen, moving in sequence from the outermost culturing zone to the innermost culturing zone; and/or
an outermost culturing zone comprising aerobic conditions, an innermost culturing zone comprising anaerobic conditions, and one or more intermediate culturing zones comprising intermediate conditions, so as to provide a gradient from aerobic conditions, decreasing in oxygen levels through to anaerobic conditions, moving in sequence from the outermost culturing zone to the innermost culturing zone;

In some embodiments of the single vessel multi-zone bioreactor, the sequence of horizontally adjacent, nested concentric cylindrical culturing zones comprises;
an innermost culturing zone of lower pH, an outermost culturing zone of higher pH, and one or more intermediate culturing zones of intermediate pH, so as to provide a gradient in pH, from lower pH to higher pH, moving in sequence from the innermost culturing zone to the outermost culturing zone; and/or
an innermost culturing zone of acidic pH, an outermost culturing zone of basic pH, and one or more intermediate culturing zones of intermediate pH, so as to provide a gradient in pH, from acidic pH to basic pH, moving in sequence from the innermost culturing zone to the outermost culturing zone; and/or
an innermost culturing zone comprising a higher partial pressure of oxygen, an outermost culturing zone comprising a lower partial pressure of oxygen, and one or more intermediate culturing zones comprising an intermediate partial pressure of oxygen, so as to provide a gradient in partial pressures of oxygen, from higher partial pressures of oxygens to lower partial pressures of oxygen, moving in sequence from the innermost culturing zone to the outermost culturing zone; and/or
an innermost culturing zone comprising aerobic conditions, an outermost culturing zone comprising anaerobic conditions, and one or more intermediate culturing zones comprising intermediate conditions, so as to provide a gradient from aerobic conditions, decreasing in oxygen levels through to anaerobic conditions, moving in sequence from the innermost culturing zone to the outermost culturing zone.

In some embodiments, the single vessel multi-zone bioreactor comprises; a gradient in pH, wherein the gradient in pH spans the range of pH 2 to pH 10, or wherein the gradient in pH spans the range of pH 4 to pH 10, or wherein the gradient in pH spans the range of pH 2 to pH 8, or wherein the gradient in pH spans the range of pH 1 to pH 7.5; and/or
a gradient in oxygen levels, wherein the gradient in oxygen levels spans the range of oxygen partial pressures of greater than 77 mmHg to less than 1 mmHg, or wherein the gradient in oxygen levels spans the range of oxygen partial pressures of 77 mmHg to 1 mmHg; and/or
a gradient in pH and a gradient in oxygen levels that simulates the gradient in human pH and oxygen levels when moving from the stomach to the rectum, thereby allowing the simultaneous culturing of multiple aerobic/anaerobic human gut microbial strains.

In some embodiments, the single vessel multi-zone bioreactor is configured to provide multiple culturing zones arranged sequentially to provide a gradient in pH wherein individual culturing zones have a pH selected from the group consisting of; 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, or 10.

In some embodiments, the single vessel multi-zone bioreactor is configured to provide multiple culturing zones arranged sequentially to provide a gradient in oxygen levels wherein individual culturing zones have an oxygen level selected from the group consisting of; 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49 and 0.5 mol/m³ of oxygen.

In some embodiments, the single vessel multi-zone bioreactor is configured to provide multiple culturing zones arranged sequentially to provide a gradient in hydrogel diffusion coefficient wherein individual culturing zones have a hydrogel diffusion coefficient [D (m²/s)] selected from the group consisting of; 0.1e-8, 0.11e-8, 0.12e-8, 0.13e-8, 0.14e-8, 0.15e-8, 0.16e-8, 0.17e-8, 0.18e-8, 0.19e-8, 0.2e-8, 0.21e-8, 0.22e-8, 0.23e-8, 0.24e-8, 0.25e-8, 0.26e-8, 0.27e-8, 0.28e-8, 0.29e-8, 0.3e-8, 0.31e-8, 0.32e-8, 0.33e-8, 0.34e-8, 0.35e-8, 0.36e-8, 0.37e-8, 0.38e-8, 0.39e-8, 0.4e-8, 0.41e-8, 0.42e-8, 0.43e-8, 0.44e-8, 0.45e-8, 0.46e-8, 0.47e-8, 0.48e-8, 0.49e-8, 0.5e-8, 0.51e-8, 0.52e-8, 0.53e-8, 0.54e-8, 0.55e-8, 0.56e-8, 0.57e-8, 0.58e-8, 0.59e-8, 0.6e-8, 0.61e-8, 0.62e-8, 0.63e-8, 0.64e-8, 0.65e-8, 0.66e-8, 0.67e-8, 0.68e-8, 0.69e-8, 0.7e-8, 0.71e-8, 0.72e-8, 0.73e-8, 0.74e-8, 0.75e-8, 0.76e-8, 0.77e-8, 0.78e-8, 0.79e-8, 0.8e-8, 0.81e-8, 0.82e-8, 0.83e-8, 0.84e-8, 0.85e-8, 0.86e-8, 0.87e-8, 0.88e-8, 0.89e-8, 0.9e-8, 0.91e-8, 0.92e-8, 0.93e-8, 0.94e-8, 0.95e-8, 0.96e-8, 0.97e-8, 0.98e-8, 0.99e-8, 1e-8, 1.01e-8, 1.02e-8, 1.03e-8, 1.04e-8, 1.05e-8, 1.06e-8, 1.07e-8, 1.08e-8, 1.09e-8, 1.1e-8, 1.11e-8, 1.12e-8, 1.13e-8, 1.14e-8, 1.15e-8, 1.16e-8, 1.17e-8, 1.18e-8, 1.19e-8, 1.2e-8, 1.21e-8, 1.22e-8, 1.23e-8, 1.24e-8, 1.25e-8, 1.26e-8, 1.27e-8, 1.28e-8, 1.29e-8, 1.3e-8, 1.31e-8, 1.32e-8, 1.33e-8, 1.34e-8, 1.35e-8, 1.36e-8, 1.37e-8, 1.38e-8, 1.39e-8, 1.4e-8, 1.41e-8, 1.42e-8, 1.43e-8, 1.44e-8, 1.45e-8, 1.46e-8, 1.47e-8, 1.48e-8, 1.49e-8, 1.5e-8, 1.51e-8, 1.52e-8, 1.53e-8, 1.54e-8, 1.55e-8, 1.56e-8, 1.57e-8, 1.58e-8, 1.59e-8, 1.6e-8, 1.61e-8, 1.62e-8, 1.63e-8, 1.64e-8, 1.65e-8, 1.66e-8, 1.67e-8, 1.68e-8, 1.69e-8, 1.7e-8, 1.71e-8, 1.72e-8, 1.73e-8, 1.74e-8, 1.75e-8, 1.76e-8, 1.77e-8, 1.78e-8, 1.79e-8, 1.8e-8, 1.81e-8, 1.82e-8, 1.83e-8, 1.84e-8, 1.85e-8, 1.86e-8, 1.87e-8, 1.88e-8, 1.89e-8, 1.9e-8, 1.91e-8, 1.92e-8, 1.93e-8, 1.94e-8, 1.95e-8, 1.96e-8, 1.97e-8, 1.98e-8, 1.99e-8, 2e-8, 2.01e-8, 2.02e-8, 2.03e-8, 2.04e-8, 2.05e-8, 2.06e-8, 2.07e-8, 2.08e-8, 2.09e-8, 2.1e-8, 2.11e-8, 2.12e-8, 2.13e-8, 2.14e-8, 2.15e-8, 2.16e-8, 2.17e-8, 2.18e-8, 2.19e-8, 2.2e-8, 2.21e-8, 2.22e-8, 2.23e-8, 2.24e-8, 2.25e-8, 2.26e-8, 2.27e-8, 2.28e-8, 2.29e-8, 2.3e-8, 2.31e-8, 2.32e-8, 2.33e-8, 2.34e-8, 2.35e-8, 2.36e-8, 2.37e-8, 2.38e-8, 2.39e-8, 2.4e-8, 2.41e-8, 2.42e-8, 2.43e-8, 2.44e-8, 2.45e-8, 2.46e-8, 2.47e-8, 2.48e-8, 2.49e-8, 2.5e-8, 2.51e-8, 2.52e-8, 2.53e-8, 2.54e-8, 2.55e-8, 2.56e-8, 2.57e-8, 2.58e-8, 2.59e-8, 2.6e-8, 2.61e-8, 2.62e-8, 2.63e-8, 2.64e-8, 2.65e-8, 2.66e-8, 2.67e-8, 2.68e-8, 2.69e-8, 2.7e-8, 2.71e-8, 2.72e-8, 2.73e-8, 2.74e-8, 2.75e-8, 2.76e-8, 2.77e-8, 2.78e-8, 2.79e-8, 2.8e-8, 2.81e-8, 2.82e-8, 2.83e-8, 2.84e-8, 2.85e-8, 2.86e-8, 2.87e-8, 2.88e-8, 2.89e-8, 2.9e-8, 2.91e-8, 2.92e-8, 2.93e-8, 2.94e-8, 2.95e-8, 2.96e-8, 2.97e-8, 2.98e-8, 2.99e-8, and 3e-8.

In some embodiments, the single vessel multi-zone bioreactor is configured to allow for, upon completion of the culturing of the multiple microbial strains;
removal and isolation of each individual microbial strain; and/or
removal and isolation of multiple microbial strains as a microbial consortium.

In some embodiments, the disclosure of the invention herein provides a method of manufacturing a food additive, supplement or medication comprising one microbial strain, or a plurality of different microbial strains; optionally wherein each microbial strain is separately encapsulated in a pharmaceutically acceptable polymer; the method comprising the steps of;
I. inoculating each culturing zone of the multi-zone bioreactor of the present invention with a different microbial strain, wherein each microbial strain is suited to the particular pH and oxygen partial pressure of the culturing zone into which it is inoculated;
II. incubating the multi-zone bioreactor inoculated with a plurality of different microbial strains at a suitable temperature and for a suitable time, and thereby simultaneously culturing a plurality of different microbial strains;
III. and then harvesting the cultured microbial strains, wherein the harvesting process comprises the steps of;
   A. removing each of the hydrogels, individually, or together, from each of the culturing zones of the multi-zone bioreactor;
      drying each of the removed hydrogels, individually, or together;
      and crushing or processing each of the dried hydrogels, individually, or together;
or
   B. removing each of the broths, individually, or together, from each of the culturing zones of the multi-zone bioreactor;
      concentrating each of the removed broths, individually, or together, via centrifugation and/or filtration;
      and drying each of the concentrated broths, individually, or together; or
   C. removing each of the hydrogels and each of the broths from each of the culturing zones of the multi-zone bioreactor;
      concentrating each of the removed broths, via centrifugation and/or filtration;
      combining each concentrated broth with its corresponding removed hydrogel, said corresponding removed hydrogel coming from the same culturing zone in which each concentrated broth was cultured;
      drying each combination of concentrated broth and corresponding removed hydrogel;
      and crushing or processing each dried combination of concentrated broth and corresponding removed hydrogel, individually or together; or
   D. removing each of the hydrogels and each of the broths from each of the culturing zones of the multi-zone bioreactor;
      concentrating each of the removed broths, individually or together, via centrifugation and/or filtration;
      combining one or more concentrated broth(s) with one or more removed hydrogel(s);
      drying each combination of concentrated broth(s) and removed hydrogel(s);
      and crushing or processing each dried combination of concentrated broth(s) and removed hydrogel(s); and
         providing a dosage form of one or more harvested, optionally encapsulated, microbial strains.

In some embodiments, of the method of manufacturing a food additive, supplement or medication, the one or more microbial strains are selected from the group consisting of; Lactobacillus rhamnosus, Lactobacillus plantarum, Lactobacillus acidophilus, Bifidobacterium lactis, Lactobacillus casei, Lactobacillus salivarius, ssp salivarius, Anaerostipes caccae, Intestinimonas butyriciproducens, Terrisporobacter glycolicus, Faecalibacterium prausnitzii, Ruminococcus broomie, Roseburia intestinalis, Alistipes indistinctus, Bacteroides salyersiae, Adlercreutzia equolifaciens, and Collinsella aerofaciens.

In some embodiments, the present disclosure provides for the use of the single vessel multi-zone bioreactor of the present invention for the production of multiple microbial strains in a single bioreactor vessel; or for the manufacture of a food additive, supplement or medication comprising a plurality of different microbial strains; or for the manufacture of a probiotic medication comprising a plurality of different microbial strains, optionally wherein the microbial strains are encapsulated in a food grade or pharmaceutically acceptable polymer.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1: Is a schematic representation of an aspect of the invention, whereby the human gut is considered to be a plug flow reactor having various compartments in sequence moving from the stomach to the rectum, and where each compartment in the sequence has lower oxygen levels and higher pH than the previous compartment. In order to simulate the human gut plug flow reactor model, porous hydrogels of decreasing porosities are arranged sequentially so as to provide a porosity gradient that enables a series of bioreactor compartments that mimic oxygen and pH conditions present in the various compartments of the human gut.

FIG. 2: Is a schematic of an embodiment of the bioreactor and method of the invention in which multiple culturing zones, each comprising a porous hydrogel (hydrogels 1, 2, 3, 4) are arranged sequentially in a geometrically parallel arrangement, with increasing porosity (moving from left to right), within a single bioreactor vessel.

FIG. 3: Is a schematic of an embodiment of the bioreactor and method of the invention in which multiple culturing zones, each comprising a porous hydrogel (hydrogels 1, 2, 3, 4) are arranged sequentially as a series of nested cylinders, with decreasing porosity (moving from the outermost cylinder to the innermost cylinder), within a single bioreactor vessel.

FIG. 4: Is a schematic of an embodiment of the bioreactor of the invention in which multiple culturing zones, each comprising a porous hydrogel are arranged sequentially as a series of nested cylinders (“disks”), with decreasing porosity (moving from the outermost cylinder to the innermost cylinder), within a single bioreactor vessel to provide a radial gradient in increasing pH and decreasing partial pressures of oxygen (moving from the outermost cylinder to the innermost cylinder).

FIG. 5: Is a series of pictures of a 3D model and a photograph (top right) of 3D printed moulds for casting porous hydrogels arranged sequentially as a series of nested cylinders in accordance with some embodiments of the present invention.

FIG. 6: Is a series of photographs depicting an embodiment of the bioreactor of the invention fabricated from stainless steel, comprising a series of nested cylinders for casting porous hydrogels (top left), a septum seal (middle left) and lid with inoculation ports (bottom left) for inoculating each separate culturing zone with a microbial strain, and specifically adapted to be utilized in an existing conventional batch fermentation bioreactor vessel apparatus (right).

FIGS. 7-12: Are SEM images of a series of porous hydrogel samples of varying porosity as used in embodiments of the invention.

FIG. 13: Is a series of images depicting tweezer pick up tests to show that water retention and toughness/Young's Modulus varies with varying porosity or pore size, and surface chemistry/composition in porous hydrogel samples of varying porosity/pore size as used in embodiments of the invention.

FIG. 14: Is a diagram of a model bioreactor in accordance with embodiments of the present invention, comprising porous CNF hydrogels cast into culturing zones 2, 3, 4 and 5 of the mould and pH buffer solutions placed into culturing zones 1 (pH=10), 4 (pH=7) and 6 & 7 (pH=4).

FIG. 15: Is a diagram of a model bioreactor in accordance with embodiments of the present invention, comprising porous CNF hydrogels cast into culturing zones 2, 3, 4 and 5 of the mould and NaOH solution placed into culturing zone 1, DI Water into culturing zone 4, and H₂SO₄ into culturing zones 6 & 7.

FIGS. 16A & 16B: Are charts of stable pH gradients observed with the model bioreactor systems described in FIGS. 14 and 15 respectively.

FIG. 16C: Is a diagram of a bioreactor in accordance with some embodiments of the present invention, comprising 10 concentrically arranged cylindrical nested hydrogel culturing zones, wherein the outer culturing zone 10 simulating the stomach is filled with highly porous hydrogel, the inner culturing zone 1 simulating the anaerobic rectum region is filled with hydrogel of low porosity and the remaining culturing zones are filled with hydrogels that decrease in porosity moving towards the center of the concentrically arranged cylindrical hydrogel bioreactor, from a maximum porosity at the outer culturing zone 10 to a minimum porosity at the central culturing zone 1.

FIG. 16D: Is a plot of 10 hydrogels (corresponding to culturing zones 1 to 10 of FIG. 16C) possessing a stepwise decrease in diffusion coefficients, in accordance with some embodiments of the invention.

FIG. 16E: Is a plot of the stable oxygen gradient in the multi-zone bioreactor in accordance with some embodiments of the present invention, with equilibrium oxygen concentrations at 60 hours varying from close to zero in zone 1 (0.05 mol/m³), simulating the rectum region of the human gut, up to 0.28 mol/m³ in zone 10, simulating the stomach region of the human gut.

FIG. 17A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Terrisporobacter glycolicus (TG).

FIG. 17B: Is a plot showing the growth curve measured as Free Amino Nitrogen consumption in mg/L, of a monoculture fermentation run immobilised on porous CNF hydrogel for Terrisporobacter glycolicus (TG).

FIG. 18A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Roseburia intestinalis (RI). Left and right traces refer to duplicate experiments performed using same media composition and inoculum at the same time using two fermenters placed on the left and right sides respectively of the fermenter control unit. FIG. 18B: Is a plot showing the growth curve measured as Free Amino Nitrogen and Glucose consumption in mg/L of a monoculture fermentation run immobilised on porous CNF hydrogel for Roseburia intestinalis (RI). Left and right traces refer to duplicate experiments performed using same media composition and inoculum at the same time using two fermenters placed on the left and right sides respectively of the fermenter control unit.

FIG. 19A: Is a series of plots showing the growth curve measured as OD at 600 nm (right axis), Dry Cell Weight (left axis) in g/L, and Glucose consumption (left axis) in g/L of a monoculture fermentation run immobilised on porous CNF hydrogel for Collinsella aerofaciens (CA). Left and right traces refer to duplicate experiments performed using same media composition and inoculum at the same time using two fermenters placed on the left and right sides respectively of the fermenter control unit.

FIG. 19B: Is a plot showing the growth curve measured as Free Amino Nitrogen and Glucose consumption in mg/L of a monoculture fermentation run immobilised on porous CNF hydrogel for Collinsella aerofaciens (CA). Left and right traces refer to duplicate experiments performed using same media composition and inoculum at the same time using two fermenters placed on the left and right sides respectively of the fermenter control unit.

FIG. 20A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Intestinimonas butyriciproducens (IB). Left and right traces refer to duplicate experiments performed using same media composition and inoculum at the same time using two fermenters placed on the left and right sides respectively of the fermenter control unit.

FIG. 20B: Is a plot showing the growth curve measured as Free Amino Nitrogen and Glucose consumption in mg/L of a monoculture fermentation run immobilised on porous CNF hydrogel for Intestinimonas butyriciproducens (IB). Left and right traces refer to duplicate experiments performed using same media composition and inoculum at the same time using two fermenters placed on the left and right sides respectively of the fermenter control unit.

FIG. 21A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Bacteroides salyersiae (BS). Left and right traces refer to duplicate experiments performed using same media composition and inoculum at the same time using two fermenters placed on the left and right sides respectively of the fermenter control unit.

FIG. 21B: Is a plot showing the growth curve measured as Free Amino Nitrogen and Glucose consumption in mg/L of a monoculture fermentation run immobilised on porous CNF hydrogel for Bacteroides salyersiae (BS). Left and right traces refer to duplicate experiments performed using same media composition and inoculum at the same time using two fermenters placed on the left and right sides respectively of the fermenter control unit.

FIG. 22A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Adlercreutzia equolifaciens (AE). Left and right traces refer to duplicate experiments performed using same media composition and inoculum at the same time using two fermenters placed on the left and right sides respectively of the fermenter control unit.

FIG. 22B: Is a plot showing the growth curve measured as Free Amino Nitrogen and Glucose consumption in mg/L of a monoculture fermentation run immobilised on porous CNF hydrogel for Adlercreutzia equolifaciens (AE). Left and right traces refer to duplicate experiments performed using same media composition and inoculum at the same time using two fermenters placed on the left and right sides respectively of the fermenter control unit.

FIG. 23A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Alistipes indistinctus (AI). Left and right traces refer to duplicate experiments performed using same media composition and inoculum at the same time using two fermenters placed on the left and right sides respectively of the fermenter control unit.

FIG. 23B: Is a plot showing the growth curve measured as Free Amino Nitrogen and Glucose consumption in mg/L of a monoculture fermentation run immobilised on porous CNF hydrogel for Alistipes indistinctus (AI). Left and right traces refer to duplicate experiments performed using same media composition and inoculum at the same time using two fermenters placed on the left and right sides respectively of the fermenter control unit.

FIG. 24A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Ruminococcus broomii (RB). Left and right traces refer to duplicate experiments performed using same media composition and inoculum at the same time using two fermenters placed on the left and right sides respectively of the fermenter control unit.

FIG. 24B: Is a plot showing the growth curve measured as Free Amino Nitrogen and Glucose consumption in mg/L of a monoculture fermentation run immobilised on porous CNF hydrogel for Ruminococcus broomii (RB). Left and right traces refer to duplicate experiments performed using same media composition and inoculum at the same time using two fermenters placed on the left and right sides respectively of the fermenter control unit.

FIG. 25A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Eubacterium rectale (ER). Left and right traces refer to duplicate experiments performed using same media composition and inoculum at the same time using two fermenters placed on the left and right sides respectively of the fermenter control unit.

FIG. 25B: Is a plot showing the growth curve measured as Free Amino Nitrogen and Glucose consumption in mg/L of a monoculture fermentation run immobilised on porous CNF hydrogel for Eubacterium rectale (ER). Left and right traces refer to duplicate experiments performed using same media composition and inoculum at the same time using two fermenters placed on the left and right sides respectively of the fermenter control unit.

FIG. 26A: Is a series of plots showing the growth curve measured as OD at 600 nm (right axis), Dry Cell Weight (left axis) in g/L, and Glucose consumption (left axis) in g/L, of a monoculture fermentation run immobilised on porous CNF hydrogel for Faecalibacterium prausnitzii (FP). Left and right traces refer to duplicate experiments performed using same media composition and inoculum at the same time using two fermenters placed on the left and right sides respectively of the fermenter control unit.

FIG. 26B: Is a plot showing the growth curve measured as Free Amino Nitrogen and Glucose consumption in mg/L of a monoculture fermentation run immobilised on porous CNF hydrogel for Faecalibacterium prausnitzii (FP). Left and right traces refer to duplicate experiments performed using same media composition and inoculum at the same time using two fermenters placed on the left and right sides respectively of the fermenter control unit.

FIG. 27A: Is an SEM image showing successful immobilisation of rod shaped cells of T. glycolicus 0.6 μm×2.0 μm in size grown on porous hydrogels.

FIG. 27B: Is an SEM image showing successful immobilisation of slightly curved rod shaped cells of I. butyriciproducens 0.4 μm×1.3 μm in size grown on porous hydrogels.

FIG. 27C: Is an SEM image showing successful immobilisation of cocci shaped cells of R. bromii 1.0 μm×1.0 μm in size grown on porous hydrogels.

FIG. 27D: Is an SEM image showing successful immobilisation of rod like cells of C. aerofaciens 0.5 μm×1.250 μm in size grown on porous hydrogels.

FIG. 27E: Is an SEM image showing successful immobilisation of rod shaped cells of A. equolifaciens 0.8 μm×1 μm in size grown on porous hydrogels.

FIG. 27F: Is an SEM image showing successful immobilisation of rod like cells of F. prausnitzii 0.5 μm×2.0 μm in size grown on porous hydrogels.

FIG. 27G: Is an SEM image showing successful immobilisation of rod like cells of E. rectale 0.9 μm×2.0 μm in size grown on porous hydrogels.

FIG. 27H: Is an SEM image showing successful immobilisation of cells of B. salyersiae 0.5 μm×2.0 μm in size grown on porous hydrogels.

FIG. 27I: Is an SEM image showing successful immobilisation of cells of R. intestinalis 0.5 μm×1.0 μm in size grown on porous hydrogels.

FIG. 27J: Is an SEM image showing successful immobilisation of rod shaped cells of A. indistinctus 0.5 μm×0.9 μm in size grown on porous hydrogels.

FIG. 28: Is a diagram of a model bioreactor in accordance with embodiments of the present invention, comprising porous CNF hydrogels cast into culturing zones 2, 3, 4 and 3 of the mould, wherein zone 1 of the bioreactor was left empty, zone 2 containing CNF/NaA hydrogel was inoculated with Ruminococcus broomii (RB), zone 3 containing CNF/NaA hydrogel was left uninoculated, and zone 4 containing CNF/NaA hydrogel was inoculated with Terrisporobacter glycolicus (TG).

FIG. 29A: Is a diagram of a model bioreactor in accordance with embodiments of the present invention, comprising porous CNF hydrogels cast into culturing zones 1 to 4 of the mould, wherein zone 1 of the bioreactor containing CNF/NaA hydrogel was inoculated with Terrisporobacter glycolicus (TG), zone 2 containing CNF/NaA hydrogel was inoculated with Ruminococcus broomii (RB), zone 3 containing CNF/NaA hydrogel was inoculated with Roseburia intestinalis (RI), and zone 4 containing CNF/NaA hydrogel was inoculated with Adlercreutzia equolifaciens (AE).

FIG. 29B: Is a diagram of a model bioreactor in accordance with embodiments of the present invention, comprising porous CNF hydrogels cast into culturing zones 1 to 4 of the mould, wherein zone 1 of the bioreactor containing CNF/NaA hydrogel was inoculated with Roseburia intestinalis (RI), zone 2 containing CNF/NaA hydrogel was inoculated with Collinsella aerofaciens (CA), zone 3 containing CNF/NaA hydrogel was inoculated with Bacteroides salyersiae (BS), and zone 4 containing CNF/NaA hydrogel was inoculated with Terrisporobacter glycolicus (TG).

FIG. 30: Is a series of Scanning Electron Microscopy images of microbial strains grown adjacently in accordance with embodiments of the invention, demonstrating effective immobilisation of the bacterial cells within the hydrogels; (1); Ruminococcus broomii (RB), (2); Terrisporobacter glycolicus (TG), (3): Roseburia intestinalis (RI), (4): Faecalibacterium prausnitzii (FP), (5): Bacteroides salyersiae (BS), and (6); Collinsella aerofaciens (CA).

FIG. 31: Is a diagram of a model bioreactor in accordance with embodiments of the present invention, comprising porous CNF hydrogels cast into culturing zones 1 to 4 of the mould, wherein zone 1 of the bioreactor containing CNF/NaA hydrogel of a minimum porosity was inoculated with Lactobacillus rhamnosus (LGG), zone 2 containing CNF/NaA hydrogel of a higher porosity than zone 1 was inoculated with Lactobacillus plantarum (LP), zone 3 containing CNF/NaA hydrogel of a higher porosity than zone 2 was inoculated with Lactobacillus acidophilus (LA) and Bifidobacterium lactis (BL), and zone 4 containing CNF/NaA hydrogel of a higher porosity than zone 3 was inoculated with Lactobacillus casei (LC), Lactobacillus salivarius (LS) and Ssp salivarius (SS).

### DEFINITIONS

Throughout this specification, unless the context requires otherwise, the word “comprise” or variations such as “comprises” or “comprising”, will be understood to imply the inclusion of a stated integer or group of integers but not the exclusion of any other integer or group of integers.

As used herein, the term “and/or” includes any and all combinations of one or more of the associated listed items.

As used herein, the term “porous” when referring to a substrate, product or material means a substrate, product or material that has accessible and interconnected voids located therein such that there exist pathways through which a fluid may pass, extending through the entire thickness of the material.

As used herein, the term “culturing media” includes any nutrients and/or solvents conventionally employed for the growth of microbes, and may also include anything else required to promote and/or sustain adequate growth of microbes, including but not limited to buffers, acids, bases, ionic species, salts, and/or gaseous species.

As used herein, the term “microbial metabolites” includes any molecular species produced by microbes during their growth, and may include molecular species in liquid form, or in solution, or in solid form, or in gaseous form, such as, but not limited to, carbon dioxide or hydrogen sulfide.

As used herein, the term “single vessel multi-zone bioreactor” will be understood throughout the specification and claims, as referring to an apparatus which is both suitable for, and capable of, producing therapeutically and/or commercially useful quantities of non-pathogenic and/or probiotic microbial consortia via simultaneous culturing of multiple microbial strains. In accordance with this definition, the term “single vessel multi-zone bioreactor” will be understood to exclude organ-on-a-chip and other microfluidics devices, including substantially 2-dimensional devices, as such devices are not capable of anything more than μml quantities of microbial cultures per day, which quantities could not possibly be regarded as therapeutically and/or commercially useful quantities of microbial cultures. Due to the purpose of the “single vessel multi-zone bioreactor” of the present invention being directed towards the production of therapeutically and/or commercially useful quantities of non-pathogenic and/or probiotic microbial consortia via simultaneous culturing of multiple microbial strains, the term “single vessel multi-zone bioreactor” will also be understood throughout the specification and claims, as excluding bioreactors that rely on the establishment of biofilms to define separate culturing zones within the multi-zone bioreactor.

As used herein, the term “gradient” includes not only a continuous gradient but also a stepwise or stratified gradient. The term “gradient” will be understood throughout the specification and claims, as requiring more than a single differential (ie; a binary differential, or single step difference) in the magnitude of one or more physical or chemical properties (including pH or oxygen level, or hydrogel porosity, or hydrogel surface hydrophilicity, or hydrogel water retention, or hydrogel Young's modulus, or hydrogel toughness), between two different culturing zones. Rather, the term “gradient” will be understood throughout the specification and claims, as requiring an increase or decrease in the magnitude of one or more physical or chemical properties (including pH or oxygen level, or hydrogel porosity, or hydrogel surface hydrophilicity, or hydrogel water retention, or hydrogel Young's modulus, or hydrogel toughness) across multiple culturing zones, observed in passing from one culturing zone to another culturing zone in a sequence of sequentially arranged culturing zones, wherein the sequence of sequentially arranged culturing zones comprises at least three culturing zones. Gradients in accordance with the present invention, as disclosed in the specification and as defined in the claims, will be understood as being established and maintained independently of, and in the absence of, the formation of any biofilms.

As used herein, the term “optimal microbial culture broth” will be understood throughout the specification and claims, as referring to a microbial culture broth that has reached a stationary phase in microbial growth, or a steady state in terms of microbial population. The person skilled in the art will understand that with fermentation time, as available nutrients become limited and waste products begin to build, population growth in the log phase of microbial growth begins to slow. When the number of dividing cells equals the number of dying cells, microbial cell growth achieves a plateau, or stationary phase. There is no overall population growth as a result of this. Competition for resources increases in less favourable settings, and cells become less metabolically active. The person skilled in the art will understand that different microbial strains will vary in the amount of time taken to reach a steady state, or stationary phase, in terms of microbial populations, and that variables including fermentation temperature, nutrient supply, pH and oxygen concentration will also have an effect on the amount of time taken to reach a steady state, or stationary phase, in terms of microbial populations. Preferably, an “optimal microbial culture broth” is one in which the Dry Cell Weight (DCW) is at least 0.3 g/L; more preferably an “optimal microbial culture broth” is one in which the Dry Cell Weight (DCW) is within the range of 0.3 to 4.0 g/L; most preferably an “optimal microbial culture broth” is one in which the Dry Cell Weight (DCW) is selected from the group consisting of; 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, 2.0 g/L, 2.1 g/L, 2.2 g/L, 2.3 g/L, 2.4 g/L, 2.5 g/L, 2.6 g/L, 2.7 g/L, 2.8 g/L, 2.9 g/L, 3.0 g/L, 3.1 g/L, 3.2 g/L, 3.3 g/L, 3.4 g/L, 3.5 g/L, 3.6 g/L, 3.7 g/L, 3.8 g/L, 3.9 g/L, and 4.0 g/L. Typically, an “optimal microbial culture broth” is one in which the Dry Cell Weight (DCW) is around 1±0.8 g/L.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

### DETAILED DESCRIPTION

Biomimetics or biomimicry is the emulation of the models, systems, and elements of nature for the purpose of solving complex human problems. Living organisms have evolved well-adapted structures and materials over geological time through natural selection. Biomimetics has given rise to new technologies inspired by biological solutions at macro and nanoscales. Humans have looked at nature for answers to problems throughout our existence. Nature has solved engineering problems such as self-healing abilities, environmental exposure tolerance and resistance, hydrophobicity, self-assembly, harnessing solar energy and the ability to fly.

The human gut is home to more than 100 trillion commensal microorganisms. The composition of these gut microbes significantly affects our health. various microbes colonize in different parts of the gut, from the stomach to the rectum. The colonization region of gut microbes depends on their oxygen sensitivity. The microbial composition changes from mostly aerobic bacteria in the mouth (pH˜7, pO₂˜atmospheric) to microaerophilic bacteria in the stomach (acidic pH˜1-4, pO₂˜77 mm Hg). In the small intestine, the pH becomes increasingly basic (pH˜5-5.5), and the oxygen level drops even more (pO₂˜33 mm Hg), so facultative anaerobic bacteria grow in this region. Further down to the colon and rectum, pH increases further, reaching values of greater than 7, and the oxygen level (i.e., pO₂) drops below 33 mm Hg and 1 mm Hg in the colon and near the rectum, respectively.

Since the human gut microbiome contains multiple strains of aerobic, microaerophilic, and anaerobic bacteria, it becomes quite essential to have all these different types of strains while developing probiotics as food additives, supplements, or for bio-therapeutic interventions. However, as these various strains need very different nutrients supply, pH, and oxygen levels, they cannot conventionally be grown in a single bioreactor. Multiple strains are generally grown in separate bioreactors, extracted, freeze-dried, and then mixed later in dry form. This process makes multi-strain probiotics-based health interventions expensive.

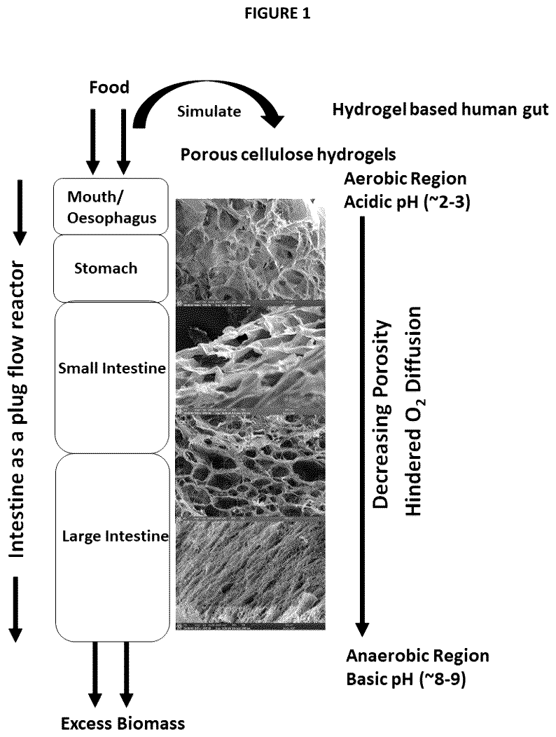

However, from a chemical engineering point of view, the gut can be treated as a compartmentalized plug flow reactor (FIG. 1). Applying such an idealized model leads to the prospect of biomimetic design of a bioreactor with various compartments interconnected, as in the human gut, that allow crosstalk between compartments and simulate realistic gastrointestinal conditions in the bioreactor, along with controlled pH and pO₂ gradients within the bioreactor to mimic human gut conditions.

In one aspect, the present invention provides a single vessel multi-zone bioreactor for simultaneously culturing multiple microbial strains comprising; multiple culturing zones, wherein; the multiple culturing zones are arranged sequentially so as to provide a gradient in pH, from lower pH to higher pH, or from acidic pH to basic pH, or from higher pH to lower pH, or from basic pH to acidic pH; and/or the multiple culturing zones are arranged sequentially so as to provide a gradient in oxygen levels, from a higher partial pressure of oxygen to a lower partial pressure of oxygen, or from aerobic conditions to anaerobic conditions, or from a lower partial pressure of oxygen to a higher partial pressure of oxygen, or from anaerobic conditions to aerobic conditions.

Once oxygen and pH gradients are established, the bioreactor can be seeded with various aerobic and anaerobic gut microbes with biotherapeutic potential. The seeding can be done with individual strains in different parts of the reactor with favourable growth environments for the corresponding strains, or multiple strains can be seeded together as a mixture. The specific microbes will then inevitably in their comfort zones of favourable environment, including pH and pO₂ values.

In one aspect, the use of immobilization materials with gradients in density or porosity is employed to establish oxygen and pH gradients in the multi-zone bioreactor of the present invention wherein the multiple culturing zones arranged sequentially each comprise, a porous hydrogel.

Organic hydrogels derived from food-grade or pharmaceutically acceptable biopolymers with tunable porosity and cross-linking density may be employed in accordance with the present invention to achieve spatial control over, and the required gradients in pH and oxygen levels.

Plant-based cellulose-derived products such as cellulose nanofibers (CNF) and cellulose nanocrystals (CNC) are commonly employed to produce organic hydrogels, and are generally modified or combined with other materials to impart desired chemical properties. CNF generally have a diameter of about 5-50 nm and fiber length can be more than 1 micron. The long aspect ratio (>100) imparts flexibility to the fibers so that it can form interconnected networks when dissolved in water, resulting in hydrogels of reasonable strength at low concentrations (<1 wt %) without external chemical crosslinking. On the other hand, CNC are rigid short crystals, which typically have low aspect ratios (<30) resulting in rigid morphology. Due to the rigid crystalline nature, CNC requires high concentrations to form hydrogels of reasonable strength. However, due to the completely different characteristics of CNF and CNC, they can be mixed in various concentration ratios to obtain hydrogels of controlled physicochemical characteristics. For example, if a highly porous hydrogel network is needed, CNF can be used. While if rigid and low porosity hydrogel is needed, CNC can be used. Therefore, regulation of the concentration and ratio of CNF and CNC in a gel system can be used to tune the porosity and rigidity of hydrogels. Surface chemistry modifications and introducing cross-linking networks are additional methods to achieve mechanically stable CNC hydrogels.

While cellulose based hydrogels are particularly preferred for immobilizing the microbial strains within the multi-zone bioreactor of the present invention and achieving control over pH and oxygen gradients, it will be appreciated by the person skilled in the art that many other porous hydrogels may be employed without departing from the spirit of the invention, as it is well established in the art that control over density, pore size and other physical attributes may be tuned by making adjustments in the formulation of such other hydrogels, including but not limited to hydrogels comprising one or more substances selected from the group consisting of; polysaccharides, cellulose, cellulose nano fibers, cellulose derivatives, methyl cellulose, alginates, dextran, hyaluronan, hyaluronates, agar, agarose, agaropectin, chitin, chitosan, gelatin, collagen, poly(lactic-co-glycolic acid), poly(e-caprolactone), poly(glycolic acid), PLA, PVA, PAM, PEG, PEGDA, PHEMA, proteins, polypeptides, biomimetic proteins, whey proteins, soy proteins, poly(lysine), elastins, elastin mimetic proteins, resilin, resilin mimetic proteins, insulin, trypsin, catalse, deoxyribonuclease, lysozymes, amyloids, ß-galactosidase, silk fibroin, fibrinogen, including pharmaceutically acceptable derivatives and/or salts of any of the aforementioned substances.

The present invention provides a principle of general application, whereby porous hydrogels of varying porosity may be used to provide oxygen and pH gradients that mimic the plug-flow bioreactor model (FIG. 1) of the human gut.

Thus, in one aspect of the invention, the single vessel multi-zone bioreactor for simultaneously culturing multiple microbial strains comprising multiple culturing zones wherein the multiple culturing zones arranged sequentially each comprise a porous hydrogel, is configured with suitably tuned hydrogel formulations such that; the porosity of the hydrogel in each culturing zone differs from the porosity of the hydrogel in each adjacent culturing zone; and/or the surface chemistry, and/or composition of the hydrogel in each culturing zone differs from the surface chemistry, and/or composition of the hydrogel in each adjacent culturing zone; and/or the water retention of the hydrogel in each culturing zone differs from the water retention of the hydrogel in each adjacent culturing zone; and/or the Young's modulus and/or the toughness of the hydrogel in each culturing zone differs from the Young's modulus and/or the toughness of the hydrogel in each adjacent culturing zone, thereby enabling the establishment of a suitable gradient in pH and/or a suitable gradient in oxygen levels to enable the culturing of multiple microbial strains in a single bioreactor vessel.

In some aspects of the invention, the necessary gradient in pH may be achieved either by the use of buffers, or acid/base systems, or any other chemical ingredient or buffering compound that can either diffuse through the hydrogels or be covalently attached to each hydrogel within each culturing zone so that the bioreactor maintains the pH gradient before, during, and after the growth of multiple microbial strains.

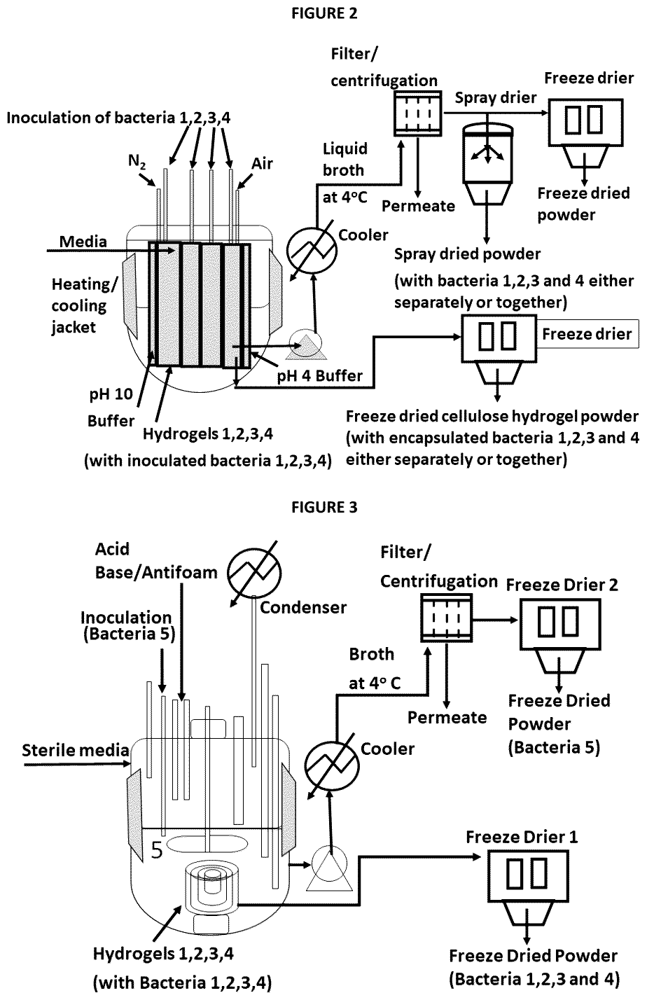

In some aspects of the invention, the necessary gradient in oxygen levels can achieved by the use of constant purging (FIG. 2) of the sequentially arranged culturing zones of the bioreactor using Nitrogen or any other suitable gas (including CO₂ or an inert gas) at one end of the sequence of sequentially arranged culturing zones, and oxygen, air (or a mixture of oxygen with an inert gas) at the opposite end of the sequence of sequentially arranged culturing zones. In some alternative aspects of the invention, oxygen producing or scavenging chemical ingredients or compounds that can either diffuse through the hydrogels or be covalently attached to each hydrogel within each culturing zone may be utilized so that the bioreactor maintains the oxygen gradient before, during and after the growth of multiple microbial strains.

With reference to FIG. 2, in some embodiments of the multi-zone bioreactor of the present invention, the sequence of culturing zones in the bioreactor may be arranged in a geometrically parallel arrangement, or in any other possible arrangement provided the requirement for providing a gradient in hydrogel porosity/pH/Oxygen levels may be satisfied, without departing from the spirit of the invention.

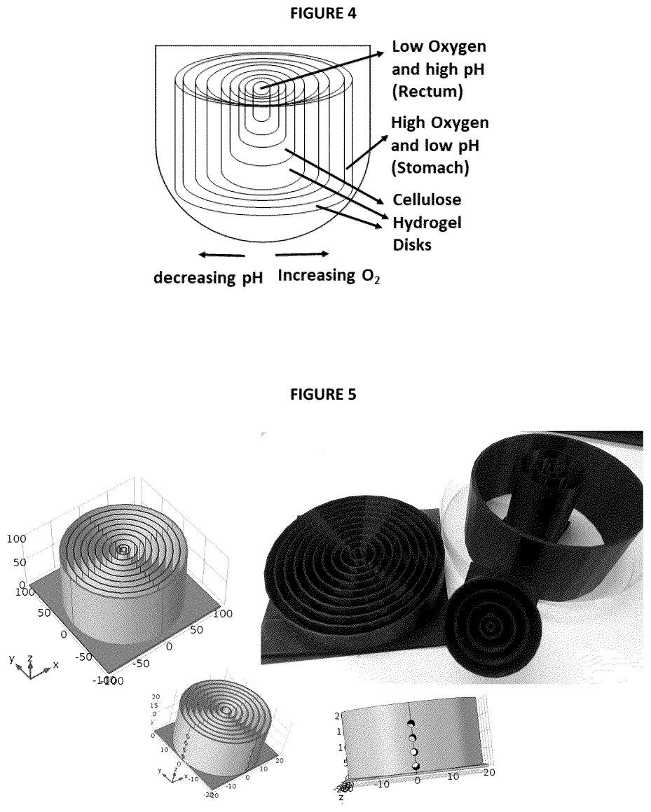

In preferred embodiments of the invention (FIGS. 3-5), the sequence of culturing zones in the bioreactor are arranged as concentric nested cylinders. Thus, in some preferred embodiments, the multiple culturing zones arranged sequentially are nested, in sequence, such that the next culturing zone in the sequence, is nested within the previous culturing zone in the sequence; or the multiple culturing zones arranged sequentially are nested, in sequence, such that the previous culturing zone in the sequence, is nested within the next culturing zone in the sequence.

The skilled addressee will appreciate that any geometric arrangement that provides a sequence of culturing zones with gradient pH/Oxygen conditions could be employed, without departing from the spirit of the invention. For example, the geometry of such nested culturing zones could be concentrically rectangular/square/triangular or any polygonal geometry in cross section (including combinations thereof), rather than the uniformly circular cross section exemplified in the aforementioned preferred embodiments.

In some embodiments of the invention, each culturing zone of the multi-zone bioreactor is separated from adjacent culturing zones by a porous membrane, capable of preventing transmigration of cultured microbial strains into adjacent culturing zones, while allowing biochemical communication between adjacent culturing zones, including allowing the transfer of culturing media and microbial metabolites between adjacent culturing zones; preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm.

In preferred embodiments of the invention, each culturing zone is separated from adjacent culturing zones by a casting mould, wherein each casting mould comprises at least one aperture (FIG. 5), allowing biochemical communication between adjacent culturing zones, including allowing the transfer of culturing media and microbial metabolites between adjacent culturing zones. The one or more apertures may comprise a porous membrane, capable of preventing transmigration of cultured microbial strains into adjacent culturing zones; most preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm.

The casting moulds may be fabricated from any sterilizable material, including, but not limited to glass, porcelain, polypropylene (PP), Teflon or any fluoropolymer, stainless steel, other metals, or any pharmaceutically acceptable or food grade polymer.

To obviate the need for apertures, the casting moulds may also be fabricated from a suitably rigid form of a porous membrane material having a suitable pore size within the ranges discussed above. For example, the casting moulds may be fabricated from a rigid cellulose acetate, or a rigid polyethersulfone (PES) material.

The casting moulds may be fabricated by any method suitable to the material from which they are made, including additive or subtractive manufacturing approaches, 3D printing, machining using CNC (Computer Numerical Control) milling (laser or any other type of milling), injection moulding, casting, welding, soldering, braising, or gluing components of one or more suitable materials together.

The casting moulds and hydrogels employed in accordance with the present invention are preferably disinfectable, most preferably serializable. The means of disinfection or sterilization may vary according to the materials used. It is only necessary to remove sufficient background microbes to eliminate their interference with, or contamination of, the desired microbes to be cultured. For example, the materials may be sterilized by UV radiation, steam, or any other suitable method. For food additives, supplements or pharmaceuticals comprising microbes cultured in accordance with the present invention, it is most preferable that the materials used may be sterilized via autoclave. In particularly preferred embodiments, the materials used are capable of being autoclaved at 121° C. for 1 hour without any deleterious effects on their properties for use in the bioreactor of the invention.

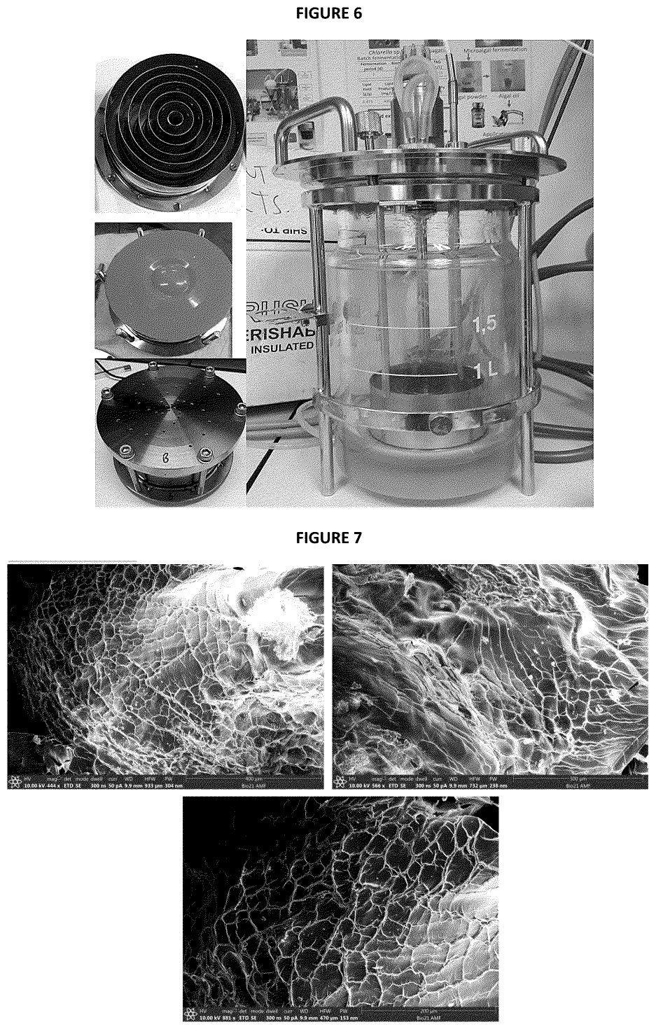

In some embodiments, the multi-zone bioreactor of the present invention is adapted to be placed inside a conventional liquid media-based batch bioreactor (FIG. 6) so that the fermentation may be conveniently carried out using existing fermentation equipment.

In conventional liquid media-based bioreactors, the single strain of probiotic bacteria grown is generally separated from liquid broth using either membrane filtration or by centrifugation. However, in the hydrogel-based gut like bioreactor of the present invention, the harvesting of multiple probiotic microbes grown can be achieved in several ways as schematically shown in FIGS. 2 & 3. As the individual microbes are grown in their own specific hydrogel culturing zone, each microbe can be harvested either separately or the consortia of microbes can be extracted together. As the microbes grow in the liquid media surrounding each hydrogel as well as immobilized on the hydrogel, they can be harvested separately.

In the first mode of microbe harvest from liquid broth conventional harvesting using filtration/centrifugation followed by drying may be employed. Advantageously, this approach also allows for culturing the microbes in semi-continuous, or fed-batch mode, whereby once the liquid broth is removed for processing, fresh liquid media of an appropriate pH and oxygen concentration may be introduced into each culturing zone, and the immobilized microbes in the hydrogel will act to inoculate the media and reinitiate the culturing process, without the need to disassemble the bioreactor.

In the second mode of harvesting microbes immobilized on hydrogels, the whole hydrogel is dried directly and then crushed. In this mode, the hydrogel immobilized microbes are advantageously encapsulated within the hydrogel matrix, protecting them from harsh gastric environments and providing a ready to use formulation for immediate preparation of dosage forms, whilst reducing the cost of probiotic production since the harvesting and encapsulation steps are combined into one step.

The third mode of harvesting is to dry the filtered liquid broth along with the hydrogels so that microbes in liquid media as well as hydrogel immobilized ones get encapsulated within the cellulose hydrogel matrix. This mode of harvesting also advantageously reduces the cost of production since the harvesting and encapsulation steps are combined into one step.

A particularly advantageous aspect of the present invention arises from the fact that probiotic microbes that favor low pH and aerobic conditions for growth (as in the stomach compartment of the human gut) are cultured in highly porous hydrogels whereas probiotic microbes that favor high pH and strictly anaerobic conditions are cultured in low porosity hydrogels. When these individual hydrogels with individual probiotic microbes are dried, the microbes become encapsulated within the respective hydrogel matrix. When subsequently deployed as probiotic dosage forms, during the transit through gastrointestinal tract, the microbes encapsulated in highly porous hydrogels are released first followed by less porous hydrogels. By tuning the porosity and pH responsiveness of the hydrogels, the controlled release of individual bacteria in the region of the gut favorable for the respective probiotic bacteria can be achieved.

Thus, in some embodiments, the present invention provides a method of manufacturing a food additive, supplement or medication comprising one microbial strain, or a plurality of different microbial strains; optionally wherein each microbial strain is separately encapsulated in a pharmaceutically acceptable polymer; the method comprising the steps of; inoculating each culturing zone of the multi-zone bioreactor of the present invention with a different microbial strain, wherein each microbial strain is suited to the particular pH and oxygen partial pressure of the culturing zone into which it is inoculated; incubating the multi-zone bioreactor inoculated with a plurality of different microbial strains at a suitable temperature and for a suitable time, and thereby simultaneously culturing a plurality of different microbial strains; and then harvesting the cultured microbial strains, wherein the harvesting process comprises the steps of;
A. removing each of the hydrogels, individually, or together, from each of the culturing zones of the multi-zone bioreactor;
   drying each of the removed hydrogels, individually, or together;
   and crushing or processing each of the dried hydrogels, individually, or together; or
B. removing each of the broths, individually, or together, from each of the culturing zones of the multi-zone bioreactor;
   concentrating each of the removed broths, individually, or together, via centrifugation and/or filtration;
   and drying each of the concentrated broths, individually, or together; or
C. removing each of the hydrogels and each of the broths from each of the culturing zones of the multi-zone bioreactor;
   concentrating each of the removed broths, via centrifugation and/or filtration;
   combining each concentrated broth with its corresponding removed hydrogel, said corresponding removed hydrogel coming from the same culturing zone in which each concentrated broth was cultured;
   drying each combination of concentrated broth and corresponding removed hydrogel;
   and crushing or processing each dried combination of concentrated broth and corresponding removed hydrogel, individually or together; or
D. removing each of the hydrogels and each of the broths from each of the culturing zones of the multi-zone bioreactor;
   concentrating each of the removed broths, individually or together, via centrifugation and/or filtration;
   combining one or more concentrated broth(s) with one or more removed hydrogel(s);
   drying each combination of concentrated broth(s) and removed hydrogel(s);
   and crushing or processing each dried combination of concentrated broth(s) and removed hydrogel(s); and
      providing a dosage form of one or more harvested, optionally encapsulated, microbial strains.

The skilled addressee will appreciate that any suitable drying method may be employed in accordance with the method of the invention, including but not limited to freeze drying, lyophilization, cryodesiccation, spray drying, supercritical drying, vacuum drying or any other food grade or pharmaceutically acceptable method of drying.

In some embodiments, the bioreactor or method of the invention may be used to culture multiple aerobic microbial strains, or multiple anaerobic microbial strains, or a combination of multiple aerobic microbial strains and multiple anaerobic microbial strains.

In some preferred embodiments, the bioreactor or method of the invention is used to culture a plurality of microbial strains selected from the group consisting of; Lactobacillus rhamnosus, Lactobacillus plantarum, Lactobacillus acidophilus, Bifidobacterium lactis, Lactobacillus casei, Lactobacillus salivarius, ssp salivarius, Anaerostipes caccae, Intestinimonas butyriciproducens, Terrisporobacter glycolicus, Faecalibacterium prausnitzii, Ruminococcus broomie, Roseburia intestinalis, Alistipes indistinctus, Bacteroides salyersiae, Adlercreutzia equolifaciens, and Collinsella aerofaciens.

Preferably, the bioreactor or method of the invention is used for the production of multiple microbial strains in a single bioreactor vessel; or for the manufacture of a food additive, supplement or medication comprising a plurality of different microbial strains; or for the manufacture of a probiotic medication comprising a plurality of different microbial strains.

The embodiments of the invention described in the preceding discussion are further illustrated by way of the following non-limiting examples.

### EXAMPLES

### Materials

pH buffers were obtained from CSA Scientific—ChemSupply Australia, with product codes BL047 (pH 4), BL048 (pH 7) and BL019 (pH 10).

Sodium hydroxide (NaOH), sulphuric acid (H₂SO₄), Calcium Chloride (CaCl₂), were obtained as analytical grade reagents from Sigma Aldrich.

Cellulose Nano Fibers (CNF), Cellulose Nano Fibers-TEMPO Oxidised (CNF-TEMPO) and Cellulose Nanocrystals (CNC) were obtained from Cellulose Lab, Canada and Sodium Alginate (NaA) was obtained from Alfa Aesar.

Microbial strains were obtained from DSMZ and ATCC.

### Characterisation

Scanning Electron Microscopy was conducted on an FEI Quanta 200.

Optical Density (OD) measurements were measured at 600 nm with a Jenaway 7300 Spectrophotometer.

Glucose estimates during fermentation runs were obtained by taking quadruplicate 2ml broth samples at regular intervals, centrifuging at 10,000 rpm for 10 minutes in a Gyrogen Mini centrifuge, collecting and filtering supernatant through 0.2μ nylon filters, and storing the supernatant samples at −20° C. prior to analysis. The analysis of the glucose concentrations of the samples was performed via HPLC (e2695 separation module, Waters®), equipped with a carbohydrate analysis HPLC column (Aminex® HPX-87H column, 300×7.8 mm) and Refractive Index detector (Waters®, 2414). Sulphuric acid (5 mM) was used as the isocratic mobile phase at a flow rate of 0.6 mL/min throughout the process at 60° C. The detection wavelength was 410 nm.

Free Amino Nitrogen (FAN) determinations were performed via the standard ninhydrin method of Wylie and Johnson.⁷ Samples collected at regular intervals were centrifuged for 3 minutes in a Gyrogen Mini centrifuge at 11,000 rpm to collect the supernatant. Colour reagent, consisting of 49.7 gL⁻¹ Na₂HPO₄·2H₂O, 60 gL⁻¹ KH₂PO₄, 5 gL⁻¹ ninhydrin and 3 gL⁻¹ fructose, was prepared. The pH of the colour reagent was monitored and fixed in between 6.6 and 6.8 by cautious addition of KH₂PO₄ during preparation. Colour reagent was stored in an amber glass bottle at 4° C. In each 15 ml glass tube, 1 ml of sample or standard solution was mixed with 0.5 ml of colour reagent. The glass tubes were sealed with a lid to prevent evaporation and then transferred into a boiling water bath for 16 minutes. After cooling for 20 minutes in an ice bath, 5 ml of dilution reagent was added to each glass tube. The dilution reagent was prepared by dissolving 2 g of KIO₃ in 200 ml DI water, which was then mixed with 384 ml of absolute ethanol before being made up to 1 L with DI water. Finally, the absorbance was measured using a spectrophotometer (UV 7300, Jenway, UK) at 570 nm by comparison with that of the standard. Samples with readings above 1.0 were diluted and reanalysed for accuracy. All quantifications were carried out in duplicate. To prepare the standard stock solution, 107.2 mg glycine was dissolved in 100 ml distilled water, and stored at 0° C. In each set of analyses triplicate samples of the standard (diluted 1:99, ie; 1 ml stock solution made up to 100 ml with distilled water) were used. The diluted standard stock solution contains 2 mg α-amino nitrogen/L. Mg/L of α-amino nitrogen is calculated via the equation: (OD of test/mean OD of standard)×2×dilution factor.

Microscopic observations for the procured microbial strains from DSMZ and ATCC were compared to the available literature. JCM catalogue has gram staining images for almost all the microbial strains used. The reference links for the microbial strains are given below. Samples were taken during fermentation at regular time intervals and after the fermentation was finished. For each sample taken at each time point, the microscopic/gram staining observation was compared to the available reference.
T. glycolicus: https://www.jcm.riken.jp/cgi-bin/jcm/jcm number?JCM=1401
R. Intestinalis: https://www.jcm.riken.jp/cgi-bin/jcm/jcm number?JCM=17583
C. aerofaciens: https://www.jcm.riken.jp/JCM/img/JCM10188B.jpg
I. Butyriciproducens: https://www.jcm.riken.jp/cgi-bin/jcm/jcm number?JCM=32124
A. equolifaciens: https://www.jcm.riken.jp/cgi-bin/jcm/jcm number?JCM=14793
F. prausnitzii: https://www.jcm.riken.jp/cgi-bin/jcm/jcm number?JCM=31915
A. indistinctus: https://www.jcm.riken.jp/cgi-bin/jcm/jcm number?JCM=16068
E. rectale: https://www.jcm.riken.jp/cgi-bin/jcm/jcm number?JCM=17463
R. bromii: https://www.microbiologyresearch.org/content/journal/ijsem/10.1099/00207713-22-2-78
B. salyersiae: https://www.jcm.riken.jp/cgi-bin/jcm/jcm number?JCM=12988
L. rhamnosus: https://www.jcm.riken.jp/cgi-bin/jcm/jcm number?JCM=2771
L. plantarum: https://www.jcm.riken.jp/cgi-bin/jcm/jcm number?JCM=1149
L. acidophilus: https://www.jcm.riken.jp/cgi-bin/jcm/jcm number?JCM=1132
B. lactis: https://www.jcm.riken.jp/cgi-bin/jcm/jcm keyword?AN=Bifidobacterium&BN=animalis&CN=lactis&DN=
L. casei: https://www.jcm.riken.jp/cgi-bin/jcm/jcm number?JCM=1177
L. salivarius: https://www.jcm.riken.jp/cgi-bin/jcm/jcm keyword?AN=Lactobacillus&BN=salivarius&CN=&DN=
Ssp, salivarius: https://www.jcm.riken.jp/cgi-bin/jcm/jcm keyword?AN=Ligilactobacillus&BN=salivarius&CN=&DN=

### Preparation of Hydrogels

A series of cellulose based hydrogels comprising Cellulose Nanofibers (CNF) and Sodium Alginate (NaA) at varying proportions, and crosslinked with Calcium Chloride (CaCl₂) were prepared to investigate trends in porosity/pore size and other properties such as water retention and toughness/Young's Modulus.

To prepare the CNF/Alginate hydrogels, the following general procedure was used. CNF in aqueous suspension was added to NaA in aqueous suspension, mixed well, cast into a petri dish and allowed to settle for 15-20 minutes. Crosslinking was then performed with the addition of aqueous CaCl₂, and the reaction was allowed to proceed for 24-48 hours before washing with DI water to remove excess CaCl₂. The sample preparations are summarised in Tables 1 and 2 below.

TABLE 1
CNF/Alginate hydrogels prepared

| Sample | SEM in | 1 wt % | NaA | CaCl₂ | |
|---|---|---|---|---|---|
| ID | FIG. | CNF (g) | (g) | (g) | DI Water (g) |
| 1 | 7 | 0.2 | 0.2 | 2 | 30 (10 + 10 + 10) |
| 2 | — | 0.2 | 0.2 | 2 | 20 (5 + 5 + 10) |
| 6 | — | 1 | 0.2 | 2 | 30 (10 + 10 + 10) |
| 7 | 8 | 0.5 | 0.2 | 2 | 30 (10 + 10 + 10) |
| 9 | — | 1 | 0.2 | 2 | 20 (5 + 5 + 10) |

With reference to Table 1, Sample ID 1, to 0.2 g of 1 wt % CNF suspended in 10 g of DI water was added 0.2 g of NaA suspended in 10 g of DI water, followed by extensive mixing (magnetic stirring). The well mixed mixture was then poured into a Petri dish followed by addition of 10 g of 20 wt % CaCl₂. The crosslinking was carried out for 24-48 hours and hydrogels were washed extensively with DI water to remove unused CaCl₂.

TABLE 2
CNF/Alginate hydrogels prepared

| Sample | SEM | wt % | wt % | wt % | Formulation |
|---|---|---|---|---|---|
| ID | FIG. | CNF | NaA | CaCl₂ | (CNF + NaA + CaCl₂) |
| 10 | — | 1 | 0.25 | 2 | 2.5 g + 2.5 g + 5 ml |
| 11 | — | 1 | 0.50 | 2 | 2.5 g + 2.5 g + 5 ml |
| 12 | — | 1 | 0.75 | 2 | 2.5 g + 2.5 g + 5 ml |
| 13 | — | 1 | 1.00 | 2 | 2.5 g + 2.5 g + 5 ml |
| 14 | — | 2 | 0.25 | 2 | 2.5 g + 2.5 g + 5 ml |
| 15 | — | 2 | 0.50 | 2 | 2.5 g + 2.5 g + 5 ml |
| 16 | — | 2 | 0.75 | 2 | 2.5 g + 2.5 g + 5 ml |
| 17 | — | 2 | 1.00 | 2 | 2.5 g + 2.5 g + 5 ml |
| 21 | 9 | 1 | 1.25 | 2 | 2.5 g + 2.5 g + 5 ml |
| 22 | 9 | 1 | 1.50 | 2 | 2.5 g + 2.5 g + 5 ml |
| 23 | 10 | 1 | 1.75 | 2 | 2.5 g + 2.5 g + 5 ml |
| 24 | 10 | 1 | 2.00 | 2 | 2.5 g + 2.5 g + 5 ml |
| 25 | 11 | 2 | 1.25 | 2 | 2.5 g + 2.5 g + 5 ml |
| 26 | 11 | 2 | 1.50 | 2 | 2.5 g + 2.5 g + 5 ml |
| 27 | 12 | 2 | 1.75 | 2 | 2.5 g + 2.5 g + 5 ml |
| 28 | 12 | 2 | 2 | 2 | 2.5 g + 2.5 g + 5 ml |

With reference to Table 2, Sample ID 10, 2.5 g of 1 wt % CNF prepared suspension in DI water was added to 2.5 g of 0.25 wt % NaA suspension in DI water, followed by mixing (magnetic stirring). The well mixed mixture was then poured into a Petri dish followed by addition of 5 ml of 2 wt % CaCl₂). The crosslinking was carried out for 24-48 hours and hydrogels were washed with DI water to remove unused CaCl₂.

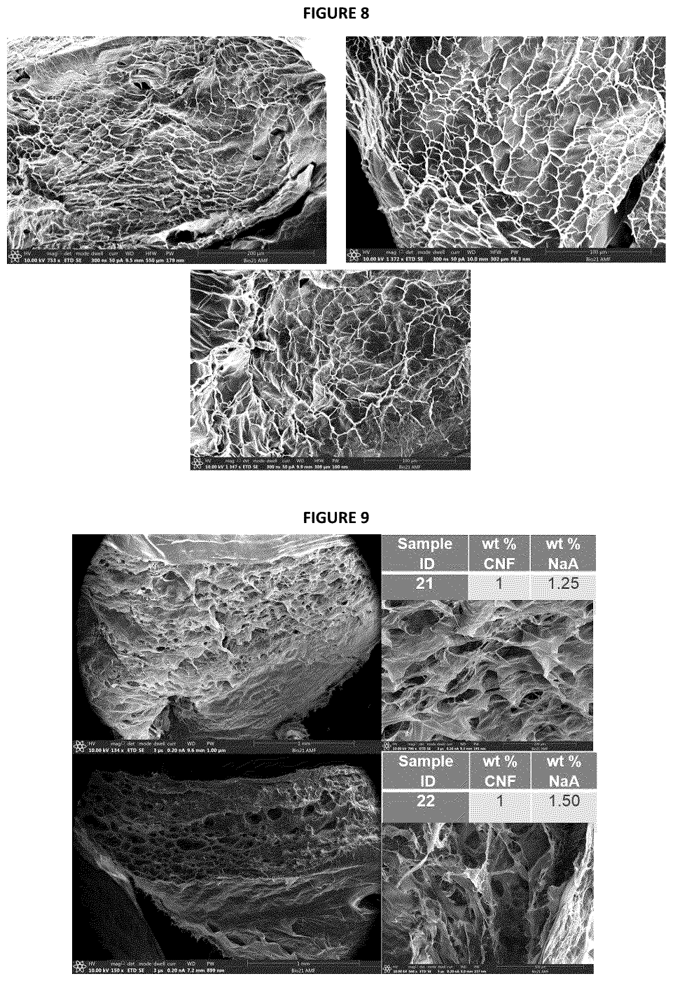

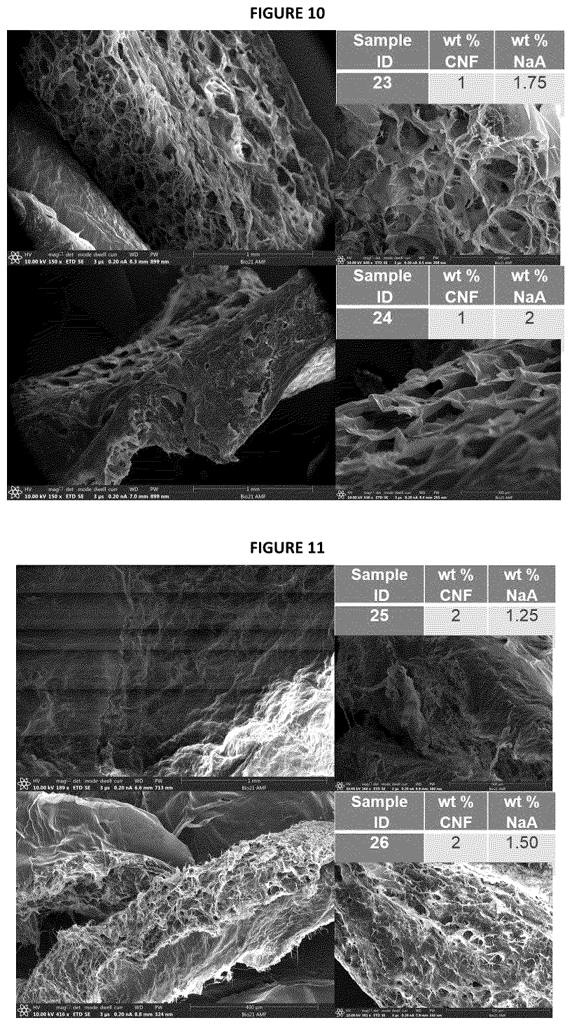

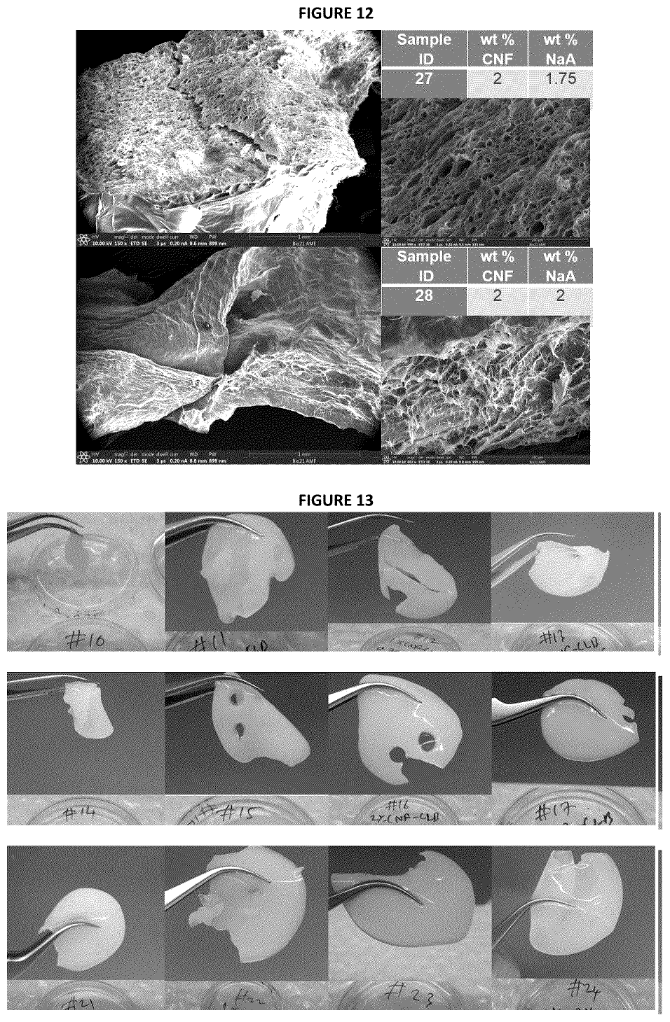

The porosities of the samples were determined via Scanning Electron Microscopy (SEM). Circular disks of about 8 mm in diameter were cut from the hydrogel for freeze drying to characterize via SEM. The results of the SEM scans are provided in FIGS. 7-12.

The toughness/Young's Modulus of each sample was investigated by picking each sample up with a pair of tweezers. Exemplary results are shown in FIG. 13.

Sample IDs 21-24, and 25-28 demonstrate that with increasing proportions of NaA relative to CNF, and the resultant increase in degree of crosslinking in the hydrogels, the porosity/pore sizes of the hydrogels decreases, (FIGS. 9-12), and the toughness/Young's Modulus increases (FIG. 13), as would be expected.

### Fabrication of Hydrogel Moulds

Exemplary hydrogel moulds for casting hydrogels to provide sequentially arranged culturing zones in accordance with some embodiments of the present invention were fabricated from 3D printed polymer (FIG. 5) and Stainless Steel (FIG. 6). 3D printing was conducted on a Creality Ender 3D printer using Poly-Lactic-Acid (PLA) as polymer filament.

The casting moulds comprise apertures allowing biochemical communication between adjacent culturing zones, including allowing the transfer of culturing media, buffers/acid/base/gases (oxygen and other gases produced by fermentation) and microbial metabolites between adjacent culturing zones, whilst preventing transmigration of cultured microbial strains into adjacent culturing zones. In the exemplary embodiments this is achieved by covering the apertures with a porous membrane, wherein the porous membrane may have a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm.

The casting moulds may be fabricated from any sterilizable material, including, but not limited to glass, porcelain, polypropylene (PP), Teflon or any fluoropolymer, stainless steel, other metals, or any pharmaceutically acceptable or food grade polymer.

To obviate the need for apertures, the casting moulds may also be fabricated from a suitably rigid form of a porous membrane material having a suitable pore size within the ranges discussed above. For example, the casting moulds may be fabricated from a rigid cellulose acetate, or a rigid polyethersulfone (PES) material.

The casting moulds may be fabricated by any method suitable to the material from which they are made, including additive or subtractive manufacturing approaches, 3D printing, machining using CNC (Computer Numerical Control) milling (laser or any other type of milling), injection moulding, casting, welding, soldering, braising, or gluing components of one or more suitable materials together.

While the geometry of the exemplary embodiments of the invention provide hydrogel moulds arranged as concentric nested cylinders, the skilled addressee will appreciate that any geometric arrangement that provides a sequence of culturing zones with gradient pH/Oxygen conditions could be employed, without departing from the spirit of the invention.

For example, the geometry of such nested culturing zones could be concentrically rectangular/square/triangular or any polygonal geometry in cross section (including combinations thereof), rather than the uniformly circular cross section exemplified herein.

The skilled addressee will also appreciate that the sequentially arranged culturing zones need not necessarily be arranged in a concentric nested fashion, but that they could also be arranged, for example, in a geometrically parallel arrangement (FIG. 2), or in any other possible arrangement provided the requirement for providing a gradient in hydrogel porosity/pH/Oxygen levels may be satisfied, without departing from the spirit of the invention.

### Establishing pH and Oxygen Gradients

Stainless steel casting moulds with seven concentrically arranged nested culturing zones (designated zones 1-7) were fabricated for testing the ability of the porous hydrogels to maintain stable pH and Oxygen gradients. Apertures between adjacent culturing zones were covered with 0.2 μm autoclavable polyethersulfone (PES) membrane filters.

Porous CNF hydrogels were prepared with compositions summarised in Table 3 below.

TABLE 3
CNF/Alginate hydrogels prepared

| Sample | wt % | wt % | wt % | FIGS. |
|---|---|---|---|---|
| ID | CNF | NaA | CaCl₂ | 14 & 15 |
| 29 | 1.5 | 2 | 2 | zone 2 |
| 30 | 1.5 | 1.5 | 2 | zone 3 |
| 31 | 1.5 | 0.75 | 2 | zone 4 |
| 32 | 1.5 | 0.5 | 2 | zone 5 |

Sample ID 29 hydrogel was prepared by combining equal amounts of 3 wt % CNF suspension with 4 wt % Sodium Alginate solution, with stirring, until the mixture becomes uniform. The hydrogel mixture was then cast into zone 2 of the bioreactor mould. An analogous approach was followed using appropriate concentrations of CNF suspensions and Sodium Alginate solutions to cast the hydrogels of Sample IDs 30-32 in zones 3-5 respectively. Excess 2 wt % CaCl₂ solution was then poured on top of the cast hydrogels and the crosslinking reaction allowed to proceed overnight before rinsing the cast, crosslinked hydrogels with deionised water to remove any residual CaCl₂ solution.

In a first example (FIG. 14), porous CNF hydrogels were cast into culturing zones 2 (Sample ID 29), 3 (Sample ID 30), 4 (Sample ID 31) and 5 (Sample ID 32) of the mould and pH buffer solutions were placed into culturing zones 1 (pH=10), 4 (pH=7, placed on top of the hydrogel), 6 and 7 (pH=4), providing a model bioreactor in accordance with embodiments of the present invention.

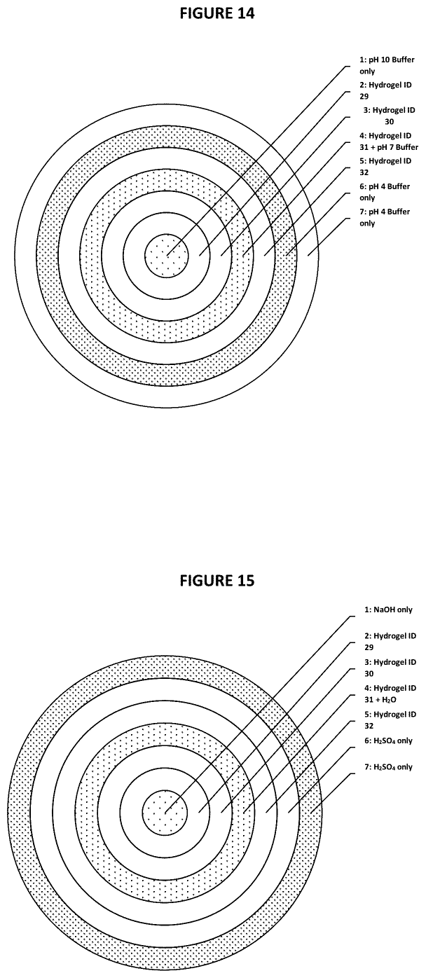

In a second example, (FIG. 15), porous CNF hydrogels were cast into culturing zones 2 (Sample ID 29), 3 (Sample ID 30), 4 (Sample ID 31) and 5 (Sample ID 32) of the mould and 0.1 mM NaOH solution was placed into culturing zone 1, DI Water into culturing zone 4 (placed on top of the hydrogel), and 0.01M H₂SO₄ into culturing zones 6 and 7, providing a model bioreactor in accordance with embodiments of the present invention.

For both examples, the model bioreactors were then autoclaved at 121° C. for 15 minutes, as they would be in normal operation, prior to inoculation, and then allowed to cool to at 37° C. (a common microbial incubation temperature). The pH of each culturing zone (corresponding to rings 1-7) was then tested.

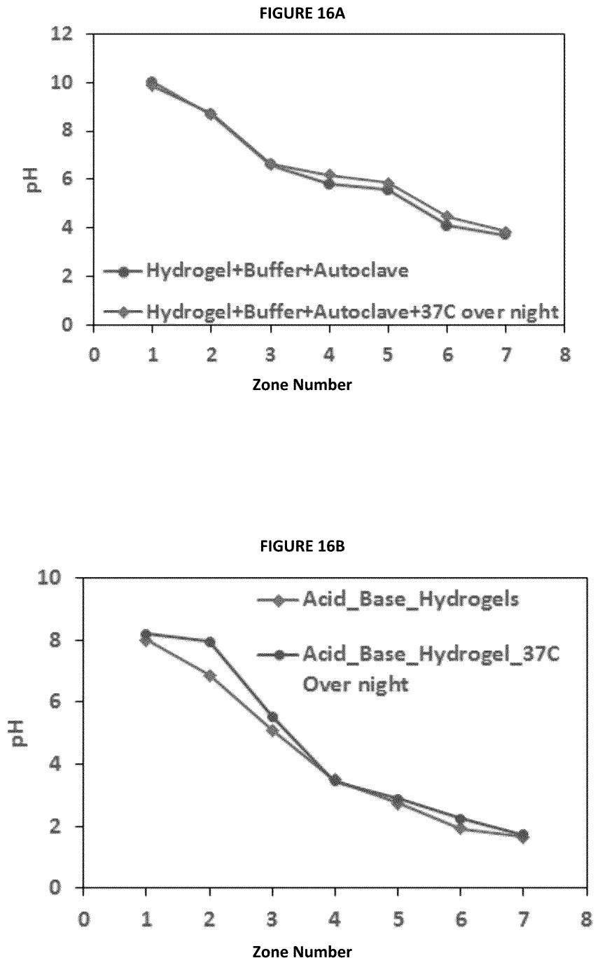

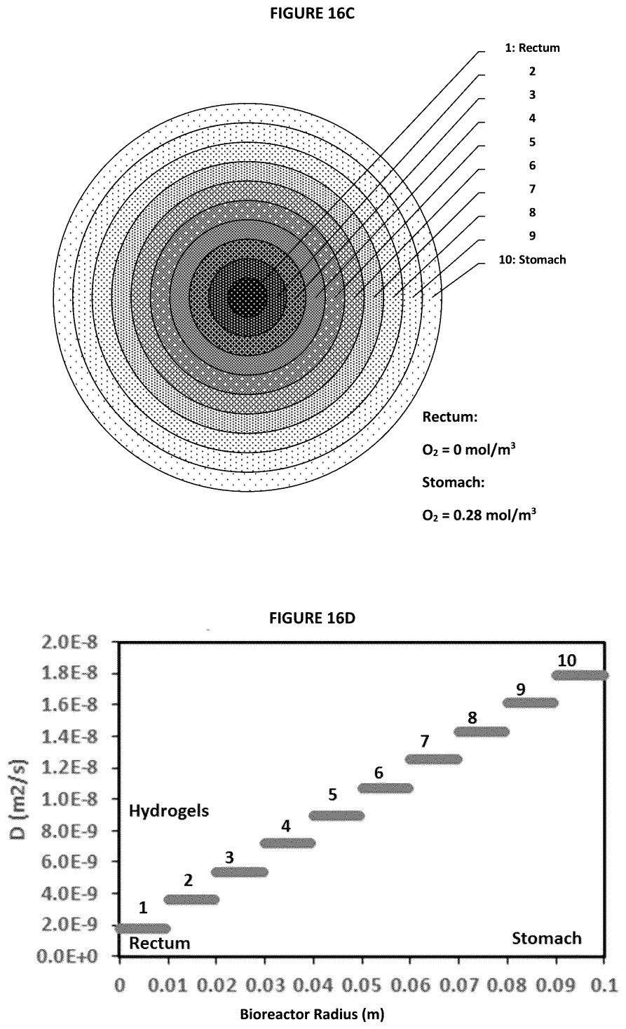

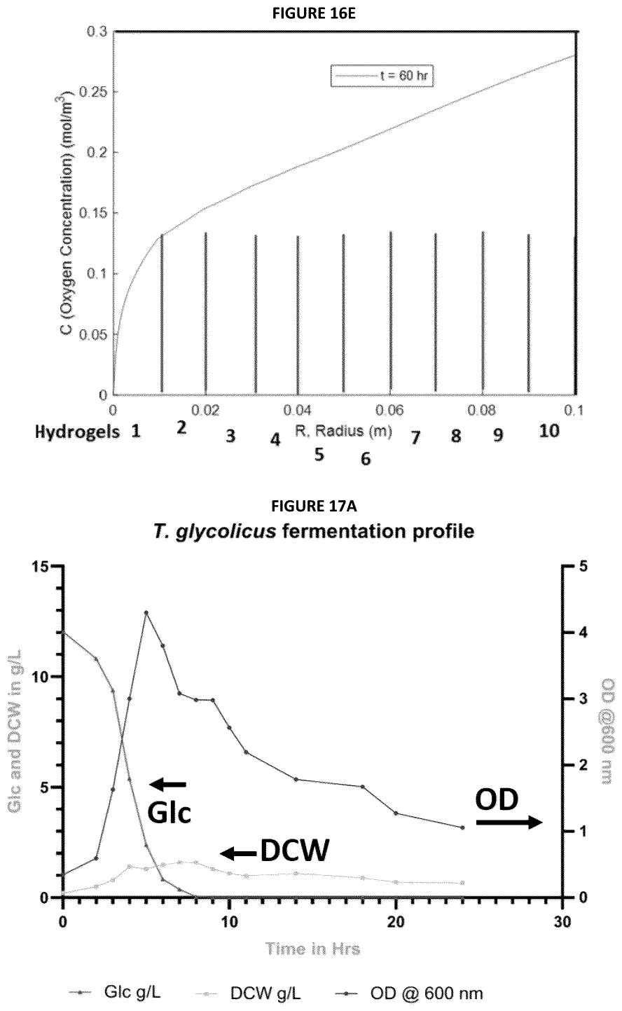

The pH of each culturing zone remained stable after the temperature of the bioreactors was maintained at 37° C. overnight. pH measurements for the first example (pH gradient established with buffering system) before and after storage at 37° C. overnight are depicted in FIG. 16A. pH measurements for the second example (pH gradient established with acid/base system) before and after storage at 37° C. overnight are depicted in FIG. 16B. The porous CNF hydrogels were able to maintain stable pH gradients from pH 10 to pH 4 (using buffer systems) or pH 8 to pH 2 (using acid/base systems).

To establish oxygen gradients in the multi-zone bioreactor of the present invention, the time dependent diffusion of oxygen into the hydrogel bioreactor can be determined using the governing equation in radial coordinates, Equation 1:

$$\frac{\partial C}{\partial t} = D [ \frac{1}{R} \frac{\partial C}{\partial R} + \frac{{\partial }^{2} C}{\partial R} ] \qquad ( 1 )$$

Where C is the mass concentration of oxygen at a given radius of the concentrically arranged cylindrical bioreactor comprising cylindrical nested culturing zones, D is the diffusion coefficient of oxygen in a particular hydrogel, t is time, and R is radial direction (R max=10 cm=0.1 m, is the Radius of the bioreactor).

As shown FIG. 16C, the concentric hydrogel bioreactor is assumed to have 10 concentric cylindrical culturing zones of thickness 1 cm each in which to cast 10 hydrogels of varying composition/porosity. The outer culturing zone 10 simulating the stomach is filled with highly porous hydrogel (low concentration of biopolymer) whereas the inner culturing zone simulating the anaerobic rectum region is filled with hydrogel of low porosity (high concentration of biopolymer). The remaining culturing zones are filled with hydrogels formulated such that the concentration of the biopolymer increases (with concomitant decrease in porosity) moving towards the center of the concentrically arranged cylindrical hydrogel bioreactor, to a maximum concentration at the central culturing zone 1.

Boundary conditions are established in the bioreactor, wherein to maintain a stable oxygen gradient in the hydrogel bioreactor, the outer culturing zone (hydrogel zone 10) that is simulating the human stomach is constantly purged with air whereas the inner culturing zone at the center of the bioreactor (hydrogel zone 1) is constantly purged with Nitrogen gas to maintain zero oxygen conditions simulating the rectum region of the human gut. Thus, the following boundary conditions can be used:

Cᵢ=0 (2)

At R=0 which is at the center of the bioreactor simulating the human rectum which is zero oxygen concentration mol/m³ at R=r min:

Cₒ=0.28 mol/m\hat{ }3 (3)

At R=R max=10 cm=0.1 m;

% At interfaces between each hydrogel culturing zone:

C_{Left}=C_{Right} (4)

Which indicates the concentration of oxygen matches at the interface between two consecutive hydrogel culturing zones; and

$${{D}_{Left} [ \frac{\partial C}{\partial R} ]}_{Left} = {{D}_{Right} [ \frac{\partial C}{\partial R} ]}_{Right} \qquad ( 5 )$$

Which indicates the concentration gradient of oxygen (flux) matches at the interface between two consecutive hydrogel culturing zones.

The diffusion Equation 1 in radial coordinates can be solved numerically for the hydrogel bioreactor once the diffusion coefficient of oxygen in the each of the 10 hydrogels of decreasing porosity is known.

Using the approximate diffusion coefficients previously reported in Figueiredo, L., et al.,⁸ a stable oxygen gradient in the hydrogel bioreactor of the present invention may be established by stacking hydrogels having a gradient in porosities moving from one adjacent culturing zone to the next.

It is well known to the skilled addressee that with increases in biopolymer concentration, the porosity of hydrogels decreases, hence the diffusion of oxygen is hindered. In other words, with an increase in biopolymer concentration in the hydrogel the diffusion coefficient of oxygen decreases. Accordingly, it is possible to tune hydrogel porosities (and therefore also to tune oxygen diffusion coefficients) in a straightforward manner, simply by adjusting the concentrations of the components when formulating.

An exemplary numerical simulation of equation 1 (in Matlab®) using the diffusion coefficients for each of 10 hydrogels possessing a stepwise decrease in diffusion coefficients (FIG. 16D) is shown in FIG. 16E. For the purposes of the model, the thickness of each hydrogel was assumed to be 1 cm=0.01 m.

FIG. 16E depicts the expected stable oxygen gradient with equilibrium oxygen concentrations at 60 hours varying from close to zero in zone 1 (0.05 mol/m³), simulating the rectum region of the human gut, up to 0.28 mol/m³ in zone 10, simulating the stomach region of the human gut.

### Gut Microbe Hydrogel Immobilisation Studies

To study the effectiveness of the porous hydrogels as immobilisation substrates for the cultivation of gut microbes, pure microbial strains were grown in separate batches (i.e. as pure culture microbial fermentations) simultaneously on three different types of porous hydrogels.

To prepare the hydrogels, a 4 wt % NaA stock solution was first prepared. 4 g of NaA was added to 96 g of DI water, and the mixture was gently heated to 50° C. with stirring to achieve a homogeneous viscous solution.

To prepare CNF/Alginate porous hydrogel, equal amounts of 3% CNF suspension in DI water and 4% NaA stock solution were combined, followed by mixing (magnetic stirring) to achieve a homogeneous hydrogel with final concentrations of CNF:Alginate of 1.5 wt % CNF:2 wt % Alginate.

To prepare CNF-TEMPO/Alginate porous hydrogel, equal amounts of 1% CNF-TEMPO suspension in DI water and 4% NaA stock solution were combined, followed by mixing (magnetic stirring) to achieve a homogeneous hydrogel with final concentrations of CNF-TEMPO:Alginate of 0.5 wt % CNF-TEMPO:2 wt % Alginate.

To prepare CNC/Alginate porous hydrogel, equal amounts of 8% CNC suspension in DI water and 4% NaA stock solution were combined, followed by mixing (magnetic stirring) to achieve a homogeneous hydrogel with final concentrations of CNC:Alginate of 4 wt % CNC:2 wt % Alginate.

The hydrogels were then cast into 10 stainless steel bioreactor moulds, each mould having three cylindrical, concentrically arranged nested culturing zones, wherein CNF/Alginate porous hydrogel was cast into the outermost culturing zone, CNF-TEMPO/Alginate porous hydrogel was cast into the middle culturing zone, and CNC/Alginate porous hydrogel was cast into the innermost culturing zone, of each of the 10 stainless steel bioreactor moulds.

Excess aqueous 2 wt % CaCl₂ was then poured on top of the casted hydrogels to initiate the cross linking reaction. The crosslinking was carried out for 24-48 hours and hydrogels were washed with DI water to remove any excess CaCl₂.

Each of the stainless steel moulds with hydrogels casted in them were placed in seperate 2L fermenter vessels (Model-Sartorius stedim 1.4435/4518), mounted with 8 ml sampling vials, pH probes (Model-Hamilton, CH-7402 Bonaduz) and placed in autoclaves along with 2M NaOH and H₂SO₄ solutions for sterilization (121° C. for 30 minutes). All the external openings were closed/connected with Masterflex-96410-16 tubes.

1.5 L batches of Reinforced Clostridial Medium (Oxoid) were autoclaved separately in 3 L Duran Bottles. These were then connected to the vessels once the autoclaving was completed, and media was poured into the vessels using peristaltic pumps.

The pH probes were connected with the Biostat-Sartorius Sedium controller systems, so that the pH throughout the fermentations could be maintained at 7 using acid and base (2M NaOH and H₂SO₄) solutions autoclaved with the vessels as described above. Fermentation temperature was maintained at 37° C. with stirring at 300 rpm. Once the setup was completed, N₂ gas at 0.2 L/Min was passed into the fermenter. The gas was sterilized through 0.2μ Sartorius PTFE filter to ensure the integrity of fermentation process was maintained, and that no contamination was introduced in the vessel.

Seed inoculum was prepared for 10 distinct strains of gut microbes in 150 ml serum bottles having 30 ml of RCM medium. Incubated at 37° C. for 24-48 hrs (time varied depending on growth characteristics of each bacteria). Gram staining was done before inoculation of seed culture in fermenter vessels and cross checked with Gram staining results of Master cultures (first inoculated in 5 ml test tubes under anaerobic conditions) and that reported in literature by the respective culture provider. Presence of anaerobic conditions in seed bottles was determined by using stock resazurin solution (1 μl/ml). If the colour of media changed from light yellow to red during the process of fermentation then it is assumed that the seed culture is not safe to proceed with inoculation as oxygen may have interfered during inoculation process.

Samples were taken at regular intervals of every 3 hrs. 8 ml of sample was taken for Dry Cell Weight (DCW) estimation (4×2 ml), samples were centrifuged at 12000 rpm for 10 mins (Gyrozen mini centrifuge). The supernatant was collected and filtered through 0.2μ syringe filters, and stored at −20° C. for analysing glucose concentration and Free Amino Nitrogen (FAN) values of samples. Optical Density (OD) was measured at 600 nm with a Jenaway 7300 Spectrophotometer.

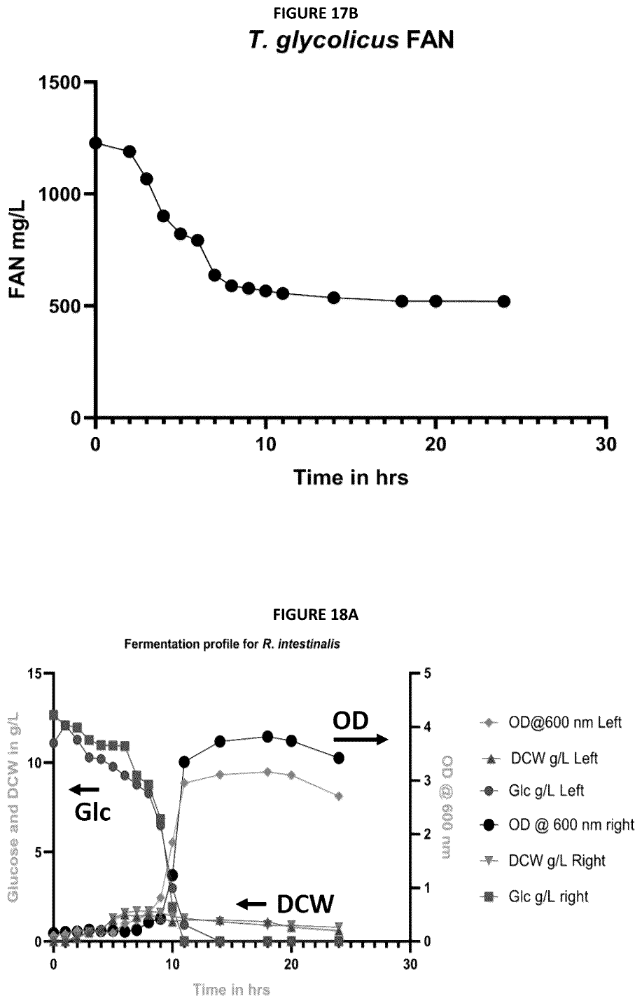

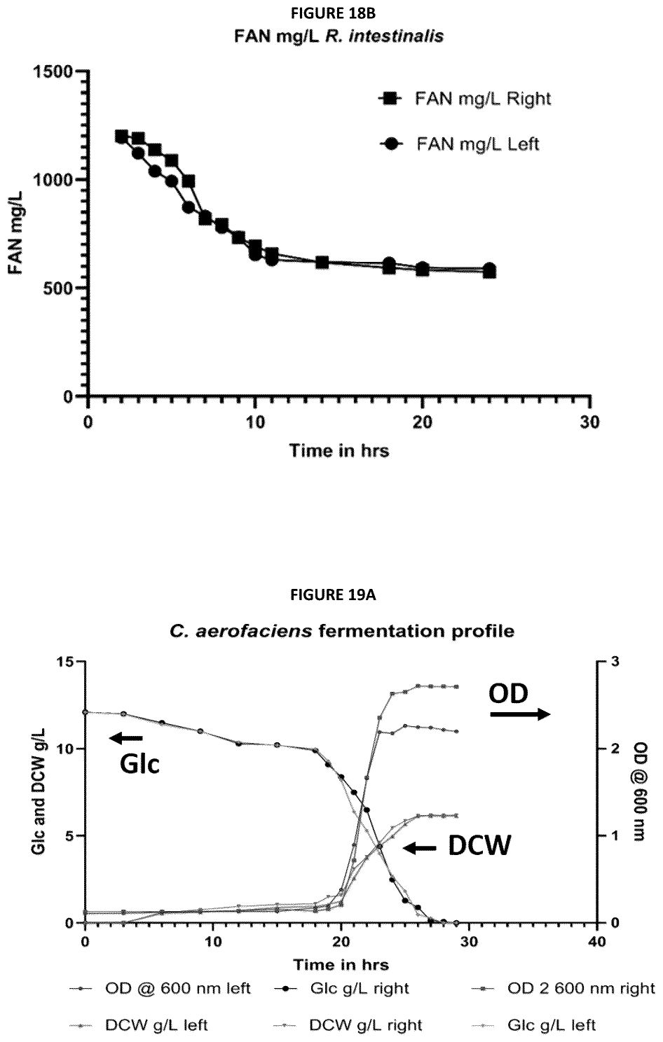

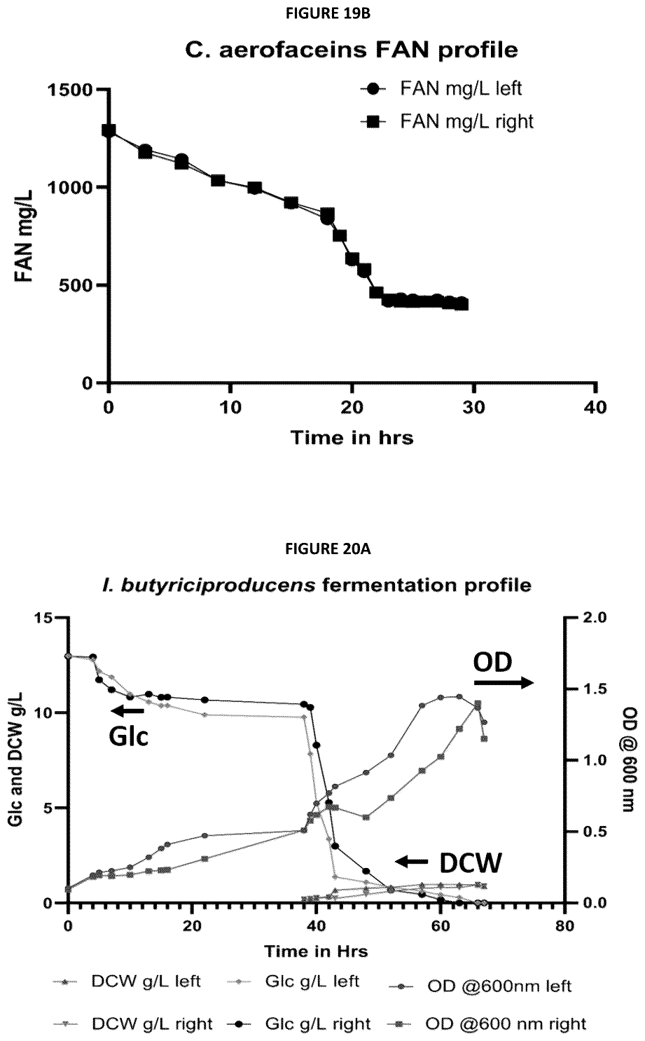

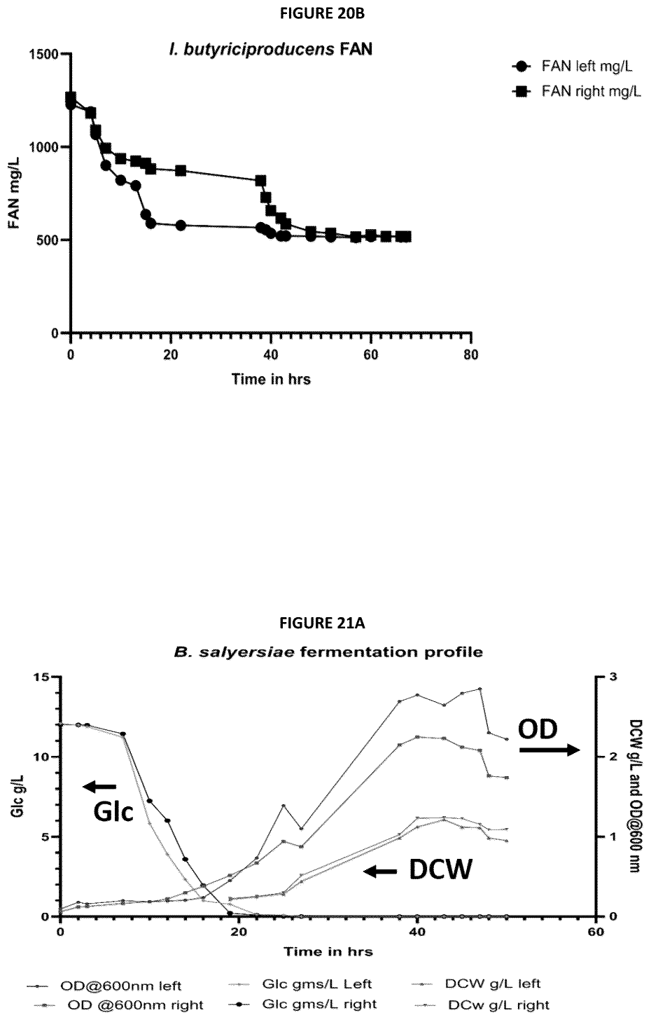

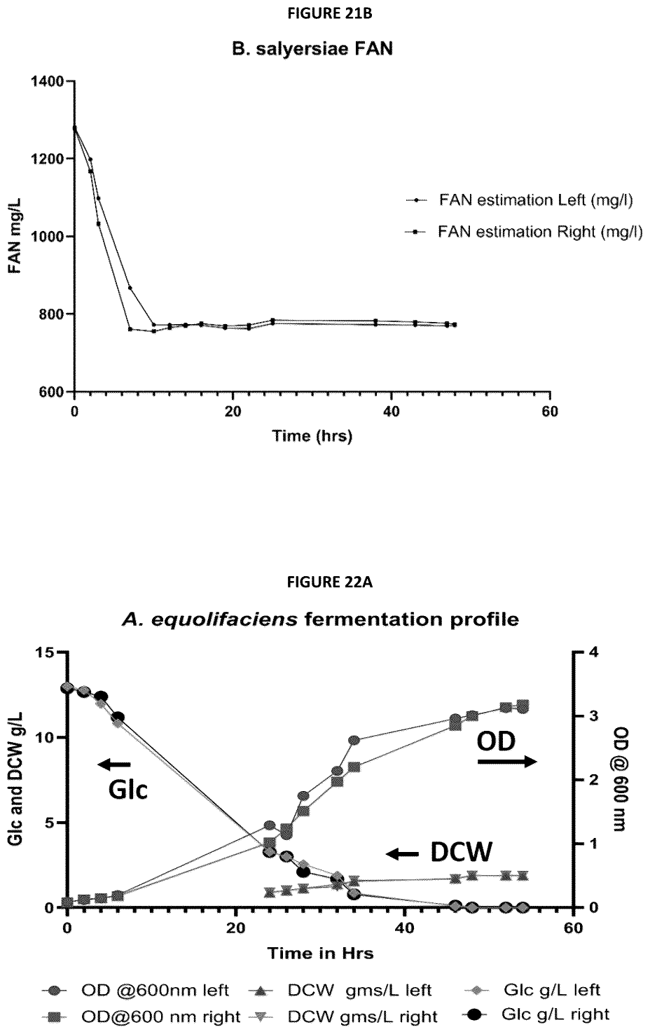

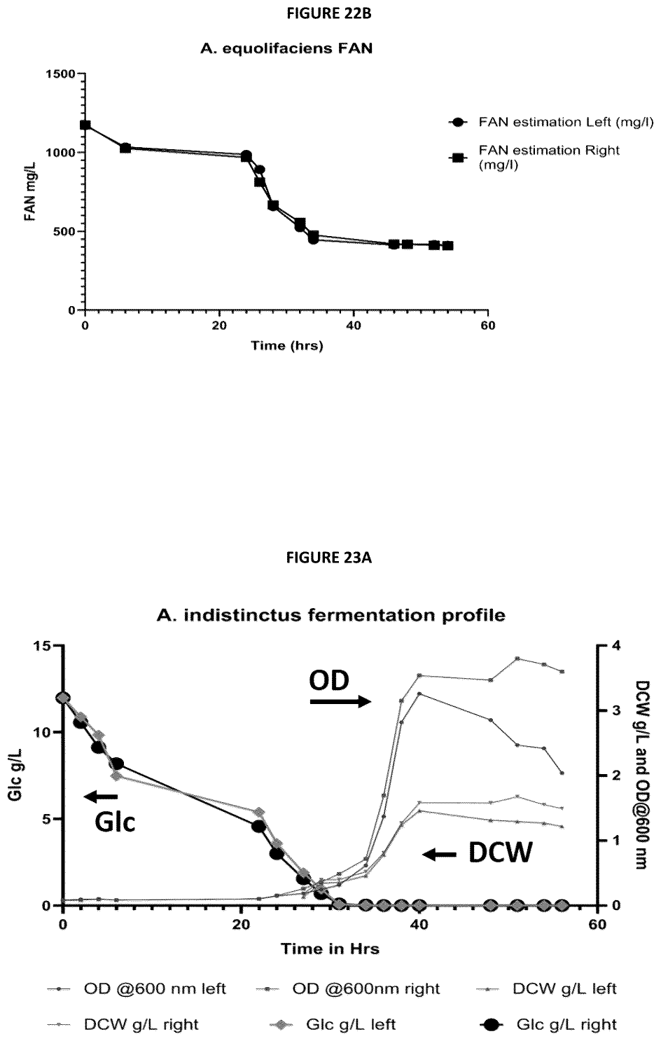

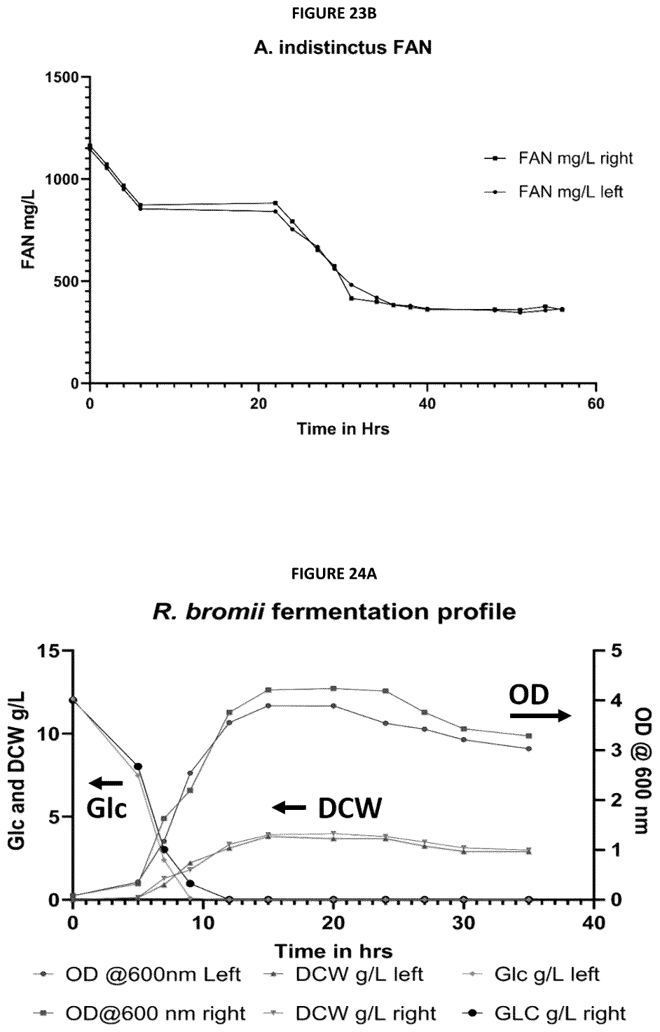

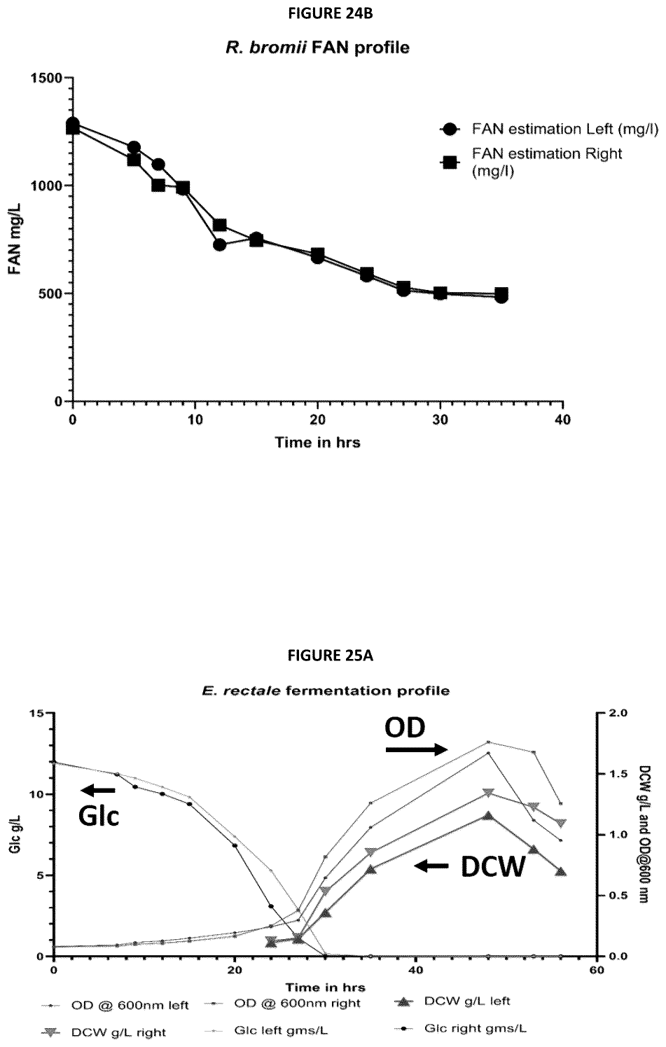

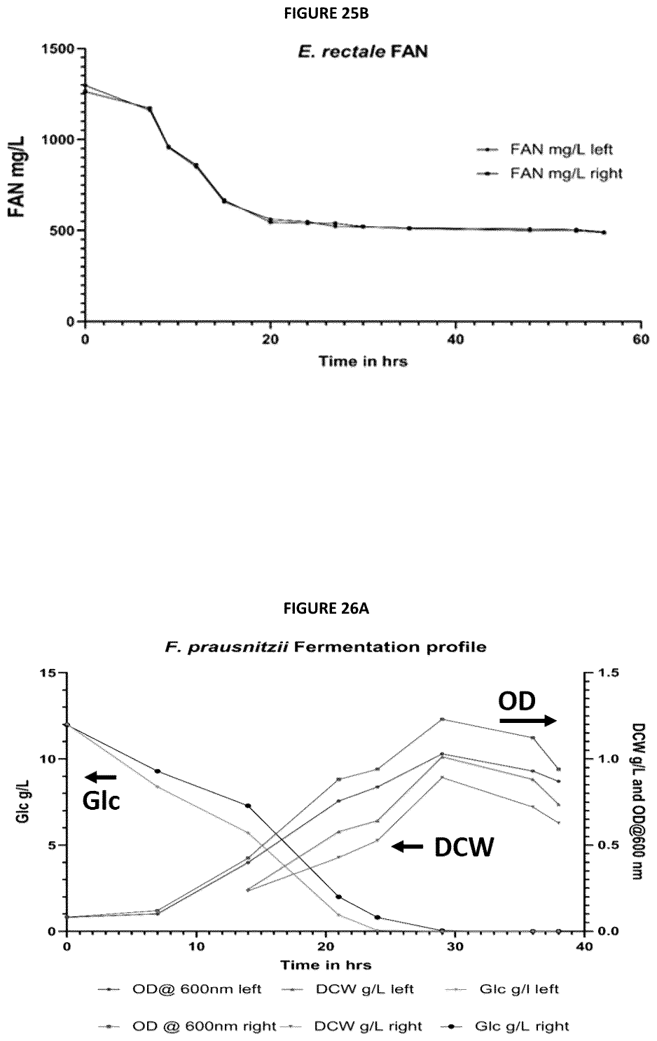

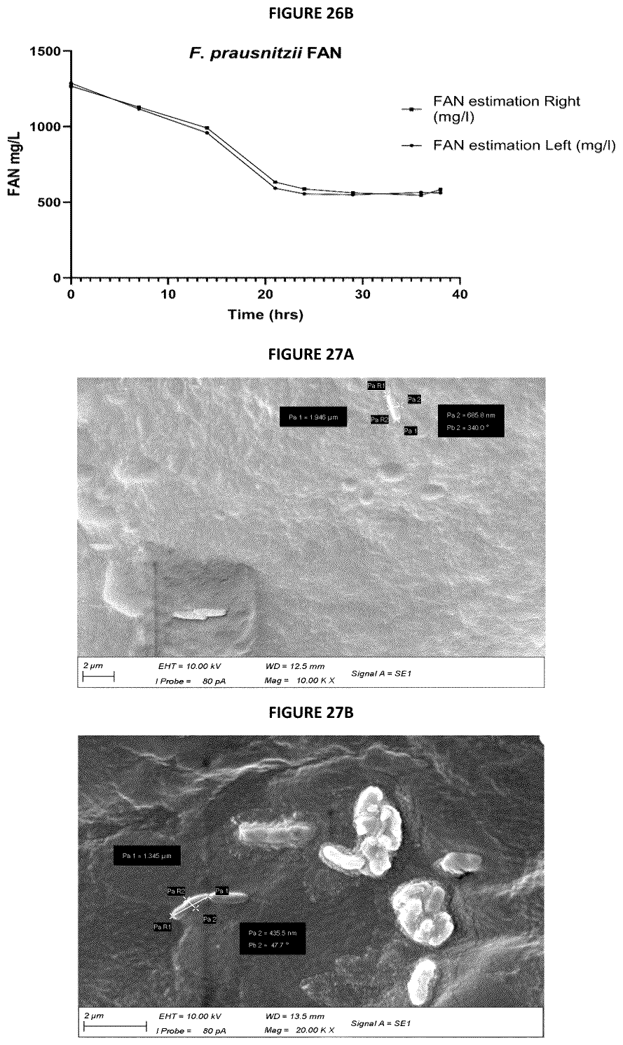

Batch fermentation with immobilisation on porous hydrogels was successfully carried out on 10 distinct strains of gut microbes: 6 strains from the Firmicutes phylum, Eubacterium rectale (ER) [FIGS. 25A and 25B], Intestinimonas butyriciproducens (IB) [FIGS. 20A and 20B], Terrisporobacter glycolicus (TG) [FIGS. 17A and 17B], Faecalibacterium prausnitzii (FP) [FIGS. 26A and 26B], Ruminococcus broomii (RB) [FIGS. 24A and 24B], Roseburia intestinalis (RI) [FIGS. 18A and 18B]; 2 strains from the Bacteroidetes phylum: Alistipes indistinctus (AI) [FIGS. 23A and 23B] and Bacteroides salyersiae (BS) [FIGS. 21A and 21B]; 2 strains from the Actinobacteria phylum: Adlercreutzia equolifaciens (AE) [FIGS. 22A and 22B] and Collinsella aerofaciens (CA) [FIGS. 19A and 19B]. Data from these batch fermentations is presented in Table A below:

TABLE A
Batch fermentation data for 10 strains of gut microbes immobilised on porous hydrogels

| | Max. OD | *Max | Time to | DCW | Specific | Max | Microscopic observation |
|---|---|---|---|---|---|---|---|
| | @600 | DCW | Max DCW | (g/L) | growth rate | FAN | Shape and Size |
| Organism/FIG. | nm | (g/L) | (hrs) | at 24 hrs | (h⁻¹) | (mg/L) | (in μ based on SEM) |
| Terrisporobacter | 4.3 | 1.6 | 8 | 0.7 | 0.343 | 707 | Gm +ve, rod |
| glycolicus (T.G) | | | | | | | 0.5μ × 2.0μ |
| FIG. 17 | | | | | | | |
| Intestinimonas | 1.4 | 1.0 | 60 | 0.3 | 0.02 | 150 | Gm −ve, rod |
| butyriciproducens | | | | | | | 0.5μ × 1.3μ |
| (I.B) | | | | | | | |
| FIG. 20 | | | | | | | |
| Ruminococcus | 4.19 | 3.9 | 20 | 3.8 | 0.135 | 722 | Gm +ve, cocci |
| bromii (R.B) | | | | | | | 1μ × 1μ |
| FIG. 24 | | | | | | | |
| Collinsella | 2.715 | 1.2 | 26 | 0.6 | 0.176 | 891 | Gm +ve, rod |
| aerofaciens (C.A) | | | | | | | 0.5μ × 1.250μ |
| FIG. 19 | | | | | | | |
| Adlercreutzia | 3 | 1.8 | 48 | 0.9 | 0.03 | 355 | Gm +ve, rod |
| equolifaciens (A.E) | | | | | | | 0.8μ × 1.0μ |
| FIG. 22 | | | | | | | |
| Faecalibacterium | 1.2 | 1.0 | 29 | 0.6 | 0.0102 | 722 | Gm −ve, rod |
| prausnitzii (F.P) | | | | | | | 0.5μ × 2.0μ |
| FIG. 26 | | | | | | | |
| Eubacterium | 1.762 | 1.3 | 48 | 0.1 | 0.142 | 773 | Gm +ve, rod |
| rectale (E.R) | | | | | | | 0.8μ × 2.0μ |
| FIG. 25 | | | | | | | |
| Bacteroides | 2.85 | 1.2 | 43 | 0.3 | 0.032 | 990 | Gm −ve, rod |
| salyersiae (B.S) | | | | | | | 0.5μ × 1.0μ |
| FIG. 21 | | | | | | | |
| Roseburia | 3.82 | 1.7 | 8 | 0.8 | 0.361 | 628 | Gm +ve, rod |
| intestinalis (R.I) | | | | | | | 0.5μ × 1.0μ |
| FIG. 18 | | | | | | | |
| Alistipes | 3.8 | 1.7 | 51 | 0.4 | 0.265 | 763 | Gm −ve, rod |
| indistinctus (A.I) | | | | | | | 0.5μ × 1.0μ |
| FIG. 23 | | | | | | | |

*Max DCW (g/L) is the Dry Cell Weight observed at the stationary phase of the fermentation

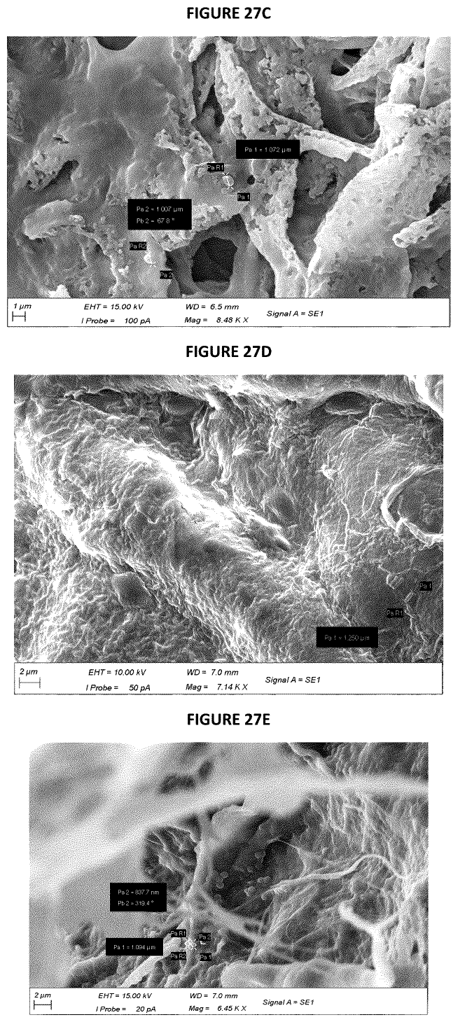

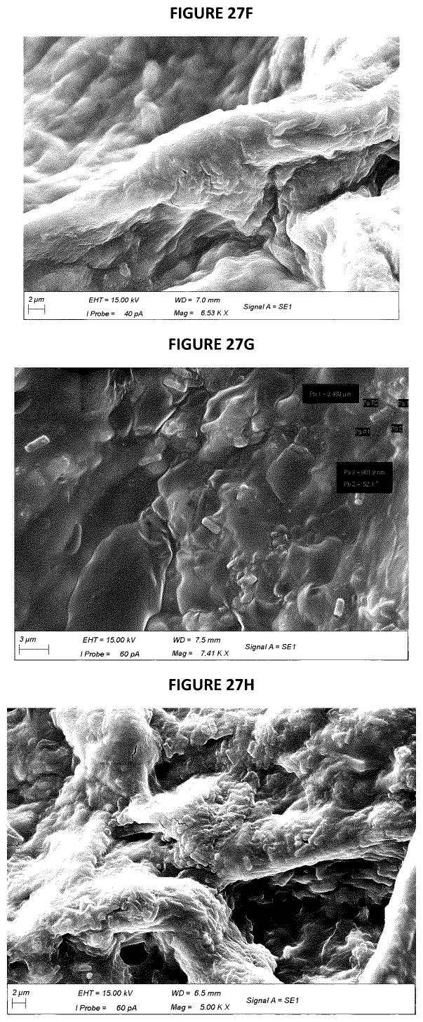

SEM analysis confirmed that the bacteria adhered very well to the porous 5 hydrogels resulting in successful immobilization (see for example FIG. 27B in the case of IB).

Samples of each of the cultured hydrogels were subjected to freeze drying, and then crushed and re-cultured on agar plates to assess viability and confirm identity. All cultured microbial strains were viable and microscopic and gram staining analysis verified the presence of pure cultures corresponding to the identity of the original seed inoculum.

### Simultaneous Culturing of Multiple Microbial Strains in a Single Bioreactor

To demonstrate the ability of the bioreactor of the present invention to simultaneously culture multiple microbial strains without cross-contamination between culturing zones, the following studies were performed.

To prepare the hydrogels for these examples, 20 g of 3% CNF suspension in DI water was added to 20 g of 2% NaA suspension in DI water, followed by mixing (magnetic stirring). The well mixed mixture was then cast into stainless steel bioreactor moulds, followed by addition of excess 2 wt % CaCl₂ on top of the casted hydrogels. The crosslinking was carried out for 24-48 hours and hydrogels were washed with DI water to remove any excess CaCl₂, to provide porous CNF 1.5 wt %:Alginate 1 wt % hydrogels.

Casting, crosslinking, setup of the fermentation apparatus and inoculation procedures were carried out in the same way as for the above-described pure culture microbial fermentations, with the following modifications.

Apertures between adjacent culturing zones in the bioreactor mould were covered with 0.2 μm autoclavable polyethersulfone (PES) membrane filters, to prevent transmigration of cultured microbial strains into adjacent culturing zones, while allowing biochemical communication between adjacent culturing zones, including allowing the transfer of culturing media and microbial metabolites between adjacent culturing zones.

Upon completion of crosslinking within each culturing zone of the bioreactor moulds and washout of excess excess CaCl₂, 10 ml of RCM media was added into each culturing zone and the top of the bioreactor was covered with a 0.2-micron cellulose acetate membrane, glued in place with high temperature silicone glue (RT 401), followed by septum rubber. The septum rubber was covered with a top plate with holes for access to each hydrogel ring. The bioreactor was sealed from the top using screws in the top plate to ensure space is left for injection of external gases, and autoclaved at 121° C. for 30 mins. After autoclaving was completed, the bioreactor was purged with nitrogen through 0.2μ PTFE filter for 10 minutes by inserting syringe needles into the access ports, through the septum rubber in the top plate.

Once the temperature of the bioreactor cooled to 40° C., each of the hydrogel culturing zones was inoculated with a separate gut bacterium via syringe needles in the access ports, through the septum rubber in the top plate.

To investigate the possibility of cross contamination between adjacent culturing zones, 2 bacterial strains were cultured on hydrogels in culturing zones adjacent to either side of a culturing zone containing an uninoculated hydrogel.

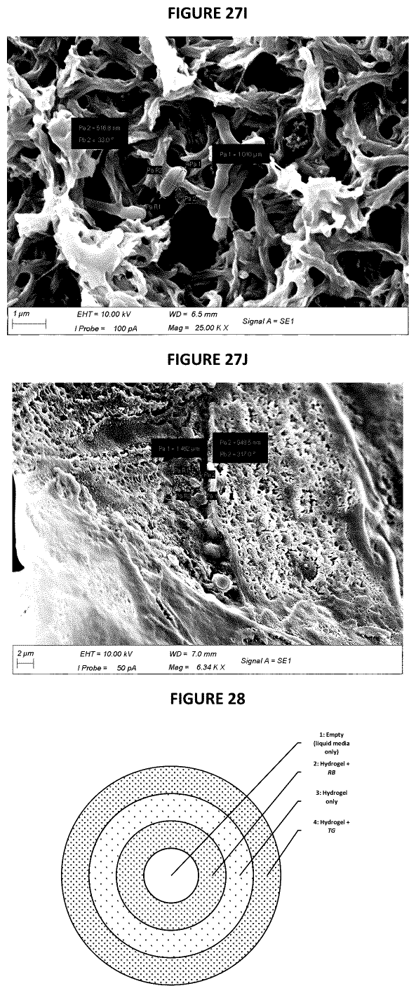

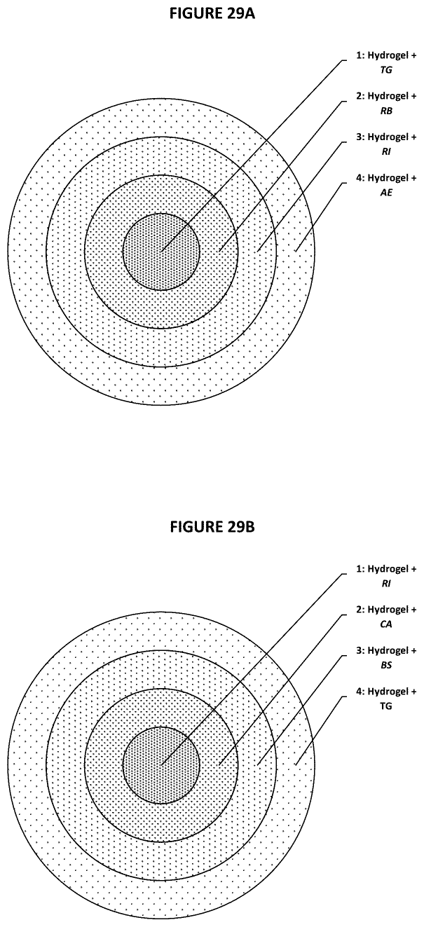

With reference to FIG. 28, zone 1 of the bioreactor was left empty, zone 2 containing CNF/NaA hydrogel was inoculated with Ruminococcus broomii (RB), zone 3 containing CNF/NaA hydrogel was left uninoculated, and zone 4 containing CNF/NaA hydrogel was inoculated with Terrisporobacter glycolicus (TG).

Duplicate fermentation runs were performed with the bioreactors incubated at 37° C. for 24 hours, and the growth as measured in terms of Optical Densities (OD at 600 nm) on completion of each run are summarised in Table 4 below.

TABLE 4
OD at 600 nm measurements from cross contamination studies

| | Zone 2: | Zone 4: |
|---|---|---|
| RUN | Ruminococcus broomii | Terrisporobacter glycolicus |
| 1 | 1.65 | 0.782 |
| 2 | 1.92 | 1.30 |

Samples of each of the three hydrogels were subjected to freeze drying, and then crushed and re-cultured on agar plates to assess viability and confirm identity. Hydrogel samples from zones 2 and 4 were viable and microscopic and gram staining analysis verified the presence of pure cultures corresponding to the identity of the original seed inoculum. The hydrogel sample from zone 3, adjacent to both of the two strains cultured, remained completely free from colonisation of either of the adjacently cultured bacterial strains.

After confirmation through gram staining and microscopic observation that cross contamination between adjacent culturing zones does not occur, 2 fermentation runs were performed where 4 anaerobic bacterial strains were cultured adjacently in a similar manner, but without separation by an uninoculated hydrogel culturing zone.

With reference to FIG. 29A, zone 1 of the bioreactor containing CNF/NaA hydrogel was inoculated with Terrisporobacter glycolicus (TG), zone 2 containing CNF/NaA hydrogel was inoculated with Ruminococcus broomii (RB), zone 3 containing CNF/NaA hydrogel was inoculated with Roseburia intestinalis (RI), and zone 4 containing CNF/NaA hydrogel was inoculated with Adlercreutzia equolifaciens (AE).

With reference to FIG. 29B, zone 1 of the bioreactor containing CNF/NaA hydrogel was inoculated with Roseburia intestinalis (RI), zone 2 containing CNF/NaA hydrogel was inoculated with Collinsella aerofaciens (CA), zone 3 containing CNF/NaA hydrogel was inoculated with Bacteroides salyersiae (BS), and zone 4 containing CNF/NaA hydrogel was inoculated with Terrisporobacter glycolicus (TG).

The bioreactors were incubated at 37° C. for 48 hours, spectrophotometric measurements confirmed that all the cultures grew steadily over a time period of 48 hrs, and the growth as measured in terms of Optical Densities (OD at 600 nm) on completion of each run are summarised in Tables 5A and 5B below. For run 1, separate OD measurements were taken from samples of the liquid broth only, and from samples of the hydrogel+liquid broth, to show that viable cells were cultured in each region of the culturing zones. For run 2, OD measurements were taken from samples of the hydrogel+liquid broth.

TABLE 5A
OD at 600 nm measurements taken for multiple
microbial strains cultured adjacently (Run 1)

| | Zone 1: | Zone 2: | Zone 3: | Zone 4: |
|---|---|---|---|---|
| | Terrisporobacter | Ruminococcus | Roseburia | Adlercreutzia |
| Sample | glycolicus | broomii | intestinalis | equolifaciens |
| Broth | 3.31 | 3.68 | 3.90 | 3.78 |
| Hydrogel + broth | 5.19 | 2.32 | 3.70 | 2.79 |

TABLE 5B
OD at 600 nm measurements taken for multiple
microbial strains cultured adjacently (Run 2)

| Zone 1: | Zone 2: | Zone 3: | Zone 4: |
|---|---|---|---|
| Roseburia | Collinsella | Bacteroides | Terrisporobacter |
| intestinalis | aerofaciens | salyers | glycolicus |
| 1.24 | 0.83 | 1.99 | 2.85 |

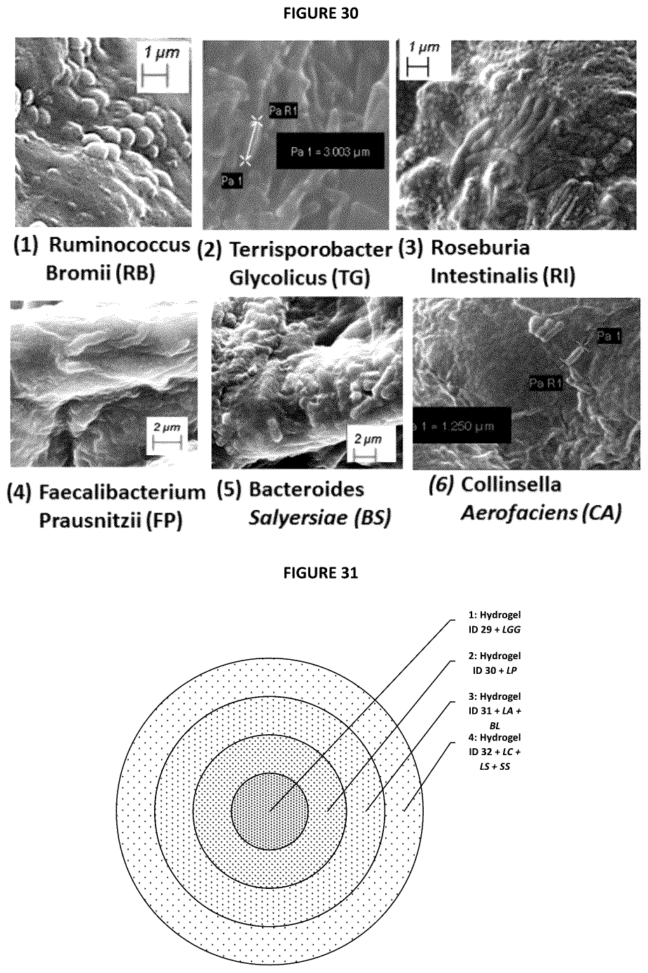

Scanning Electron Microscopy analysis of each of the six microbial strains grown adjacently in accordance with these examples demonstrated effective immobilisation of the bacterial cells within the hydrogels [FIGS. 30(1)-30(6)].

Duplicate samples (1 g) of each of the four hydrogels from each run were taken. One of each of the samples was subjected to freeze drying, and then crushed and re-cultured on agar plates to assess viability and confirm identity. The other of each of the samples was stored at −20° C. in glycerol, prior to re-culturing on agar plates to assess viability and confirm identity. The frozen samples remained stable and viable after 3 months. The results of Gram staining and microscopic observations indicated no cross contamination between culturing zones.

An analogous fermentation run was performed where 7 common probiotic aerobic bacterial strains were cultured adjacently in a similar manner, to that described above. In this example, some of the culturing zones of the bioreactor were inoculated with multiple microbial strains, and the culturing zones were cast with porous hydrogels of increasing porosity, moving from the outermost culturing zone (maximum porosity) to the innermost culturing zone (minimum porosity).

With reference to FIG. 31, zone 1 of the bioreactor containing CNF/NaA hydrogel (Table 3, Sample ID: 29) was inoculated with Lactobacillus rhamnosus (LGG), zone 2 containing CNF/NaA hydrogel (Table 3, Sample ID: 30) was inoculated with Lactobacillus plantarum (LP), zone 3 containing CNF/NaA hydrogel (Table 3, Sample ID: 31) was inoculated with Lactobacillus acidophilus (LA) and Bifidobacterium lactis (BL), and zone 4 containing CNF/NaA hydrogel (Table 3, Sample ID: 32) was inoculated with Lactobacillus casei (LC), Lactobacillus salivarius (LS) and Ssp salivarius (SS). The culturing media used in this example was MRS.

The bioreactor was incubated at 37° C. for 24 hours, spectrophotometric measurements confirmed that all the cultures grew steadily over a time period of 24 hrs.

The above examples demonstrate the ability of the bioreactor of the present invention to simultaneously culture multiple microbial stains in a single vessel, without cross-contamination between culturing zones.

### General

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

It should be appreciated that throughout this specification, any reference to any prior publication, including prior patent publications and non-patent publications, is not an acknowledgment or admission that any of the material contained within the prior publication referred to was part of the common general knowledge as at the priority date of the application.

Any manufacturer's instructions, descriptions, product specifications, and product sheets for any products mentioned herein or in any document incorporated by reference herein, are hereby incorporated herein by reference, and may be employed in the practice of the invention.

The invention described herein may include one or more range of values (eg. size, displacement and field strength etc). A range of values will be understood to include all values within the range, including the values defining the range, and values adjacent to the range which lead to the same or substantially the same outcome as the values immediately adjacent to that value which defines the boundary to the range.

The present invention is not to be limited in scope by any of the specific embodiments described herein. These embodiments are intended for the purpose of exemplification only. Functionally equivalent products, formulations and methods are clearly within the scope of the invention as described herein.

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

### REFERENCES

1. Blanton, L. V.; Barratt, M. J.; Charbonneau, M. R.; Ahmed, T.; Gordon, J. I., Childhood undernutrition, the gut microbiota, and microbiota-directed therapeutics. Science 2016, 352 (6293), 1533-1533.
2. Costello, E. K.; Relman, D. A., Immaturity in the gut microbial community. Nature 2014, 510 (7505), 344-345.
3. Gehrig, J. L.; Venkatesh, S.; Chang, H.- W.; Hibberd, M. C.; Kung, V. L.; Cheng, J.; Chen, R. Y.; Subramanian, S.; Cowardin, C. A.; Meier, M. F., Effects of microbiota-directed foods in gnotobiotic animals and undernourished children. Science 2019, 365 (6449), eaau4732.
4. Pennisi, E., Gut microbes may help malnourished children. American Association for the Advancement of Science: 2019.
5. Raman, A. S.; Gehrig, J. L.; Venkatesh, S.; Chang, H.-W.; Hibberd, M. C.; Subramanian, S.; Kang, G.; Bessong, P. O.; Lima, A. A.; Kosek, M. N., A sparse covarying unit that describes healthy and impaired human gut microbiota development. Science 2019, 365 (6449), eaau4735.
6. Subramanian, S.; Huq, S.; Yatsunenko, T.; Haque, R.; Mahfuz, M.; Alam, M. A.; Benezra, A.; DeStefano, J.; Meier, M. F.; Muegge, B. D.; Barratt, M. J.; VanArendonk, L. G.; Zhang, Q.; Province, M. A.; Petri Jr, W. A.; Ahmed, T.; Gordon, J. I., Persistent gut microbiota immaturity in malnourished Bangladeshi children. Nature 2014, 510 (7505), 417-421.
7. Wylie, E. B., and Johnson, M. J. Biochim, Biophys. Acta, 59:450, 1962.1975, rev. 1976, 2010.
8. Figueiredo, L., et al., (Assessing glucose and oxygen diffusion in hydrogels for the rational design of 3D stem cell scaffolds in regenerative medicine. Journal of tissue engineering and regenerative medicine, 2018. 12(5): p. 1238-1246).

1. A single vessel multi-zone bioreactor for simultaneously culturing multiple microbial strains comprising multiple culturing zones; wherein the multiple culturing zones are arranged sequentially to provide a sequence of at least three culturing zones; wherein the multiple culturing zones each comprise, a porous hydrogel, and a liquid culturing media; and wherein;
a) the multiple culturing zones are arranged sequentially so as to provide a gradient in pH, from lower pH to higher pH, or from acidic pH to basic pH, or from higher pH to lower pH, or from basic pH to acidic pH; and/or
b) the multiple culturing zones are arranged sequentially so as to provide a gradient in oxygen levels, from a higher partial pressure of oxygen to a lower partial pressure of oxygen, or from aerobic conditions to anaerobic conditions, or from a lower partial pressure of oxygen to a higher partial pressure of oxygen, or from anaerobic conditions to aerobic conditions.

2. The single vessel multi-zone bioreactor of claim 1, wherein the single vessel multi-zone bioreactor is adapted to be operated in batch culture mode, or fed-batch culture mode, or continuous culture mode, to produce at least 100 ml per day per culturing zone of optimal microbial culture broth.

3. The single vessel multi-zone bioreactor of claim 1, wherein;
a) the porosity of the hydrogel in each culturing zone differs from the porosity of the hydrogel in each adjacent culturing zone; and/or
b) the surface chemistry, and/or composition of the hydrogel in each culturing zone differs from the surface chemistry, and/or composition of the hydrogel in each adjacent culturing zone; and/or
c) the water retention of the hydrogel in each culturing zone differs from the water retention of the hydrogel in each adjacent culturing zone; and/or
d) the Young's modulus and/or the toughness of the hydrogel in each culturing zone differs from the Young's modulus and/or the toughness of the hydrogel in each adjacent culturing zone.

4. The single vessel multi-zone bioreactor of claim 3, wherein;
a) the porosity of the hydrogel in each culturing zone differs from the porosity of the hydrogel in each adjacent culturing zone, such that the sequence of sequentially arranged culturing zones provides a hydrogel porosity gradient; and/or
b) the surface chemistry of the hydrogel in each culturing zone differs from the surface chemistry of the hydrogel in each adjacent culturing zone, such that the sequence of sequentially arranged culturing zones provides a hydrogel surface hydrophilicity gradient; and/or
c) the hydrophilicity and water retention of the hydrogel in each culturing zone differs from the water retention of the hydrogel in each adjacent culturing zone, such that the sequence of sequentially arranged culturing zones provides a hydrogel water retention gradient; and/or
d) the Young's modulus and/or the toughness of the hydrogel in each culturing zone differs from the Young's modulus and/or the toughness of the hydrogel in each adjacent culturing zone, such that the sequence of sequentially arranged culturing zones provides a hydrogel Young's modulus and/or hydrogel toughness gradient.

5. The single vessel multi-zone bioreactor of claim 4, wherein;
I. the hydrogel porosity gradient comprises;
a) an increasing porosity gradient, where the porosity of the hydrogel in each culturing zone is greater than the porosity of the hydrogel in the previous culturing zone in the sequence of sequentially arranged culturing zones; or
b) a decreasing porosity gradient, where the porosity of the hydrogel in each culturing zone is less than the porosity of the hydrogel in the previous culturing zone in the sequence of sequentially arranged culturing zones;
and/or wherein;
II. the hydrogel surface hydrophilicity gradient comprises;
a) an increasing hydrogel surface hydrophilicity gradient, where the hydrophilicity of the hydrogel in each culturing zone is greater than the hydrophilicity of the hydrogel in the previous culturing zone in the sequence of sequentially arranged culturing zones; or
b) a decreasing hydrogel surface hydrophilicity gradient, where the hydrophilicity of the hydrogel in each culturing zone is less than the hydrophilicity of the hydrogel in the previous culturing zone in the sequence of sequentially arranged culturing zones;
and/or wherein;
III. the hydrogel water retention gradient comprises;
a) an increasing hydrogel water retention gradient, where the water retention of the hydrogel in each culturing zone is greater than the water retention of the previous culturing zone in the sequence of sequentially arranged culturing zones; or
b) a decreasing hydrogel water retention gradient, where the water retention of the hydrogel in each culturing zone is less than the water retention of the previous culturing zone in the sequence of sequentially arranged culturing zones.

6. The single vessel multi-zone bioreactor of claim 1, wherein the multiple culturing zones arranged sequentially comprise porous hydrogels, wherein the porous hydrogels comprise one or more substances selected from the group consisting of; polysaccharides, cellulose, cellulose nano fibers, cellulose derivatives, methyl cellulose, alginates, dextran, hyaluronan, hyaluronates, agar, agarose, agaropectin, chitin, chitosan, gelatin, collagen, poly(lactic-co-glycolic acid), poly(e-caprolactone), poly(glycolic acid), PLA, PVA, PAM, PEG, PEGDA, PHEMA, proteins, polypeptides, biomimetic proteins, whey proteins, soy proteins, poly(lysine), elastins, elastin mimetic proteins, resilin, resilin mimetic proteins, insulin, trypsin, catalase, deoxyribonuclease, lysozymes, amyloids, β-galactosidase, silk fibroin, fibrinogen, including pharmaceutically acceptable derivatives and/or salts of any of the aforementioned substances.

7. The single vessel multi-zone bioreactor of claim 1, wherein;
a) the multiple culturing zones arranged sequentially are nested, in sequence, such that the next culturing zone in the sequence, is nested within the previous culturing zone in the sequence; or
b) the multiple culturing zones arranged sequentially are nested, in sequence, such that the previous culturing zone in the sequence, is nested within the next culturing zone in the sequence.

8. The single vessel multi-zone bioreactor of claim 1, wherein each culturing zone is separated from adjacent culturing zones by a porous membrane, capable of preventing transmigration of cultured microbial strains into adjacent culturing zones, while allowing biochemical communication between adjacent culturing zones, including allowing the transfer of culturing media and microbial metabolites between adjacent culturing zones; preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm.

9. The single vessel multi-zone bioreactor of claim 1, wherein each culturing zone is separated from adjacent culturing zones by a casting mould, wherein each casting mould comprises at least one aperture, allowing biochemical communication between adjacent culturing zones, including allowing the transfer of culturing media and microbial metabolites between adjacent culturing zones; optionally wherein;
a) the apertures comprise a porous membrane, capable of preventing transmigration of cultured microbial strains into adjacent culturing zones; most preferably wherein the porous membrane has a pore size within the range of 150 to 0.5 μm, or within the range of 100 to 0.01 μm, or within the range of 1.0 to 0.01 μm, preferably within the range of 0.5 to 0.1 μm, most preferably wherein the pore size is approximately 0.2 μm; and/or
b) the casting moulds are fabricated from a sterilizable material such as but not limited to glass, porcelain, polypropylene (PP), Teflon or any fluoropolymer, stainless steel, other metals, or any pharmaceutically acceptable or food grade polymer; and/or
c) the casting moulds are manufactured via 3D printing.

10. The single vessel multi-zone bioreactor of claim 1, wherein the single vessel is a cylindrical vessel, and the multiple culturing zones of the bioreactor comprise a sequence of horizontally adjacent, nested concentric cylindrical culturing zones.

11. The single vessel multi-zone bioreactor of claim 10, wherein;
I. the sequence of horizontally adjacent, nested concentric cylindrical culturing zones comprises;
a) an outermost culturing zone of lower pH, an innermost culturing zone of higher pH, and one or more intermediate culturing zones of intermediate pH, so as to provide a gradient in pH, from lower pH to higher pH, moving in sequence from the outermost culturing zone to the innermost culturing zone; and/or
b) an outermost culturing zone of acidic pH, an innermost culturing zone of basic pH, and one or more intermediate culturing zones of intermediate pH, so as to provide a gradient in pH, from acidic pH to basic pH, moving in sequence from the outermost culturing zone to the innermost culturing zone; and/or
c) an outermost culturing zone comprising a higher partial pressure of oxygen, an innermost culturing zone comprising a lower partial pressure of oxygen, and one or more intermediate culturing zones comprising an intermediate partial pressure of oxygen, so as to provide a gradient in partial pressures of oxygen, from higher partial pressures of oxygen to lower partial pressures of oxygen, moving in sequence from the outermost culturing zone to the innermost culturing zone; and/or
d) an outermost culturing zone comprising aerobic conditions, an innermost culturing zone comprising anaerobic conditions, and one or more intermediate culturing zones comprising intermediate conditions, so as to provide a gradient from aerobic conditions, decreasing in oxygen levels through to anaerobic conditions, moving in sequence from the outermost culturing zone to the innermost culturing zone;
or;
II. the sequence of horizontally adjacent, nested concentric cylindrical culturing zones comprises;
a) an innermost culturing zone of lower pH, an outermost culturing zone of higher pH, and one or more intermediate culturing zones of intermediate pH, so as to provide a gradient in pH, from lower pH to higher pH, moving in sequence from the innermost culturing zone to the outermost culturing zone; and/or
b) an innermost culturing zone of acidic pH, an outermost culturing zone of basic pH, and one or more intermediate culturing zones of intermediate pH, so as to provide a gradient in pH, from acidic pH to basic pH, moving in sequence from the innermost culturing zone to the outermost culturing zone; and/or
c) an innermost culturing zone comprising a higher partial pressure of oxygen, an outermost culturing zone comprising a lower partial pressure of oxygen, and one or more intermediate culturing zones comprising an intermediate partial pressure of oxygen, so as to provide a gradient in partial pressures of oxygen, from higher partial pressures of oxygens to lower partial pressures of oxygen, moving in sequence from the innermost culturing zone to the outermost culturing zone; and/or
d) an innermost culturing zone comprising aerobic conditions, an outermost culturing zone comprising anaerobic conditions, and one or more intermediate culturing zones comprising intermediate conditions, so as to provide a gradient from aerobic conditions, decreasing in oxygen levels through to anaerobic conditions, moving in sequence from the innermost culturing zone to the outermost culturing zone.

12. The single vessel multi-zone bioreactor of claim 1, comprising;
a) a gradient in pH, wherein the gradient in pH spans the range of pH 2 to pH 10, or wherein the gradient in pH spans the range of pH 4 to pH 10, or wherein the gradient in pH spans the range of pH 2 to pH 8, or wherein the gradient in pH spans the range of pH 1 to pH 7.5; and/or
b) a gradient in oxygen levels, wherein the gradient in oxygen levels spans the range of oxygen partial pressures of greater than 77 mmHg to less than 1 mmHg, or wherein the gradient in oxygen levels spans the range of oxygen partial pressures of 77 mmHg to 1 mmHg; and/or
c) a gradient in pH and a gradient in oxygen levels that simulates the gradient in human body pH and oxygen levels when moving from the stomach to the rectum, thereby allowing the simultaneous culturing of multiple aerobic/anaerobic human gut microbial strains.

13. The single vessel multi-zone bioreactor of claim 1, wherein the bioreactor is configured to allow for, upon completion of the culturing of the multiple microbial strains;
a) removal and isolation of each individual microbial strain; and/or
b) removal and isolation of multiple microbial strains as a microbial consortium.

14. A method of manufacturing a food additive, supplement or medication comprising a plurality of different microbial strains; optionally wherein each microbial strain is separately encapsulated in a pharmaceutically acceptable polymer; the method comprising the steps of;
I. inoculating each culturing zone of the multi-zone bioreactor of claim 1 with a different microbial strain; wherein each microbial strain is suited to the particular pH and oxygen partial pressure of the culturing zone into which it is inoculated; wherein, upon inoculation of each culturing zone with a different microbial strain, and through to completion of the culturing process, each liquid culturing media within each culturing zone may be referred to as a broth;
II. incubating the multi-zone bioreactor inoculated with a plurality of different microbial strains at a suitable temperature and for a suitable time, and thereby simultaneously culturing a plurality of different microbial strains;
III. and then harvesting the cultured microbial strains, wherein the harvesting process comprises the steps of;
A. removing each of the hydrogels, individually, or together, from each of the culturing zones of the multi-zone bioreactor;
drying each of the removed hydrogels, individually, or together;
and crushing or processing each of the dried hydrogels, individually, or together; or
B. removing each of the broths, individually, or together, from each of the culturing zones of the multi-zone bioreactor;
concentrating each of the removed broths, individually, or together, via centrifugation and/or filtration;
and drying each of the concentrated broths, individually, or together; or
C. removing each of the hydrogels and each of the broths from each of the culturing zones of the multi-zone bioreactor;
concentrating each of the removed broths, via centrifugation and/or filtration;
combining each concentrated broth with its corresponding removed hydrogel, said corresponding removed hydrogel coming from the same culturing zone in which each concentrated broth was cultured;
drying each combination of concentrated broth and corresponding removed hydrogel;
and crushing or processing each dried combination of concentrated broth and corresponding removed hydrogel, individually or together; or
D. removing each of the hydrogels and each of the broths from each of the culturing zones of the multi-zone bioreactor;
concentrating each of the removed broths, individually or together, via centrifugation and/or filtration;
combining one or more concentrated broth(s) with one or more removed hydrogel(s);
drying each combination of concentrated broth(s) and removed hydrogel(s);
and crushing or processing each dried combination of concentrated broth(s) and removed hydrogel(s); and
providing a dosage form of one or more harvested, optionally encapsulated, microbial strains.

15. The method of claim 14 wherein the microbial strains are selected from the group consisting of; Lactobacillus rhamnosus, Lactobacillus plantarum, Lactobacillus acidophilus, Bifidobacterium lactis, Lactobacillus casei, Lactobacillus salivarius, ssp salivarius, Anaerostipes caccae, Intestinimonas butyriciproducens, Terrisporobacter glycolicus, Faecalibacterium prausnitzii, Ruminococcus broomie, Roseburia intestinalis, Alistipes indistinctus, Bacteroides salyersiae, Adlercreutzia equolifaciens, and Collinsella aerofaciens.

16. Use of the single vessel multi-zone bioreactor of claim 1;
a) for the production of multiple microbial strains in a single bioreactor vessel; or
b) for the manufacture of a food additive, supplement or medication comprising a plurality of different microbial strains; or
c) for the manufacture of a probiotic medication comprising a plurality of different microbial strains.
